SEQUENCE LISTING (4 sequences)

SEQ ID NO: 1 - DNA, 21 bp, Artificial Sequence
  PCR Primer
gcgtccacccgcgagtacaac

SEQ ID NO: 2 - DNA, 20 bp, Artificial Sequence
  PCR Primer
cgacgacgagcgcagcgata

SEQ ID NO: 3 - DNA, 22 bp, Artificial Sequence
  PCR Primer
tactgtgtgcctcctcggtctt

SEQ ID NO: 4 - DNA, 23 bp, Artificial Sequence
  PCR Primer
agctcgagtgactgacaggaatc

(19) United States
(12) Patent Application Publication
(10) Pub. No.: US 2015/0080380 A1
(43) Pub. Date: Mar. 19, 2015

(54) Therapeutic Uses

(71) Applicant: GLAXOSMITHKLINE INTELLECTUAL PROPERTY DEVELOPMENT LIMITED, Brentford, Middlesex (GB)

(72) Inventors: Hongtao Lu, Pudong (CN); Rong Wang, Pudong (CN)

(21) Appl. No.: 14/372,055

(22) PCT Filed: Jan. 15, 2013
(86) PCT No.: PCT/CN2013/070489

§ 371 Date: Jul. 14, 2014

(30) Foreign Application Priority Data
Jan. 16, 2012 (WO) PCTCN2012070438

(51) Int. Cl. A61K 31/55; A61K 31/501; A61K 31/4453; A61K 31/454; A61K 31/4545

(52) U.S. Cl. CPC A61K 31/55; A61K 31/454; A61K 31/4545; A61K 31/4453; A61K 31/501
USPC 514/217.01; 514/321; 514/316; 514/317; 514/252.05

(57) ABSTRACT

This invention relates to novel therapeutic uses for compounds which are inverse agonists of the H3 receptor. In particular this invention relates to therapeutic use of these compounds in the treatment of Multiple Sclerosis.

This invention relates to novel therapeutic uses of compounds which are inverse agonists of the H3 receptor. In particular this invention relates to therapeutic use of these compounds in the treatment of Multiple Sclerosis.

### BACKGROUND TO THE INVENTION

Multiple Sclerosis (MS) is a chronic inflammatory, demyelinating disease that affects the central nervous system. The fundamental injury in MS is the inflammatory mediated demyelination of axons in the CNS which is thought to be caused by altered immune system function. Demyelination can result in reduced trophic support for axons, redistribution of ion channels, and destabilization of action potential membrane potentials. Axons can initially adapt, but eventually distal and retrograde degeneration occurs leading to subsequent development of disability.

There is growing evidence that an endogenous CNS repair mechanism exists to combat demyelinating events during the course of MS. This process of remyelination is mediated by a precursor cell population referred to as oligodendrocyte precursor cells (OPCs). Post-mortem data and experimental studies point to the failure of OPC differentiation as the main cause of remyelination failure in MS. Therefore, the early promotion of remyelination through OPC differentiation and preservation of oligodendrocytes is an important target in MS. Following a demyelinating event, OPCs are rapidly recruited and amplified in areas of demyelination where they give rise to myelinating oligodendrocytes. As lesions evolve, there is prominent astrocytic proliferation (gliosis). Surviving oligodendrocytes or those that differentiate from precursor cells may partially remyelinate the surviving naked axons, producing so-called shadow plaques. Although remyelination in response to primary demyelination is well documented and can be surprisingly efficient in a subset of individuals, it often fails during the course of MS for reasons not fully understood. As a result, fully remyelinated lesions are comparatively rare. Instead, one will find that in many lesions, remyelination does not fail entirely, but remain restricted to a small rim of newly formed myelin sheaths at the lesions border. Furthermore, it has been shown that in many lesions, OPCs are present in large numbers but fail to differentiate, supporting the hypothesis that enhancement of OPC differentiation is a viable target for remyelination in MS (Kotter et al Enhancing remyelination in disease—can we wrap it up? Brain (2011) 134: 1882-1990).

In the early stages of MS, inflammatory attacks occur over short intervals of acutely heightened disease activity. These episodes are followed by periods of recovery and remission. During the remission period, the local swelling in the nervous system lesion resolves, the immune cells become less active or inactive, and the myelin-producing cells remyelinate the axons. Nerve signalling improves, and the disability caused by the inflammation and demyelination becomes less severe or goes away entirely. This phase of the disease is called relapsing-remitting MS (RRMS). The lesions do not all heal completely, though. Some remain as “chronic” lesions, which usually have a demyelinated core region which lacks immune cells. Over time, the neurons in the center of such lesions mostly die, although inflammation often continues at their edges. The brain can adapt well to the loss of some neurons, and permanent disability may not occur for many years. However, more than 50% of patients with MS eventually enter a stage of progressive deterioration, called secondary progressive MS (SPMS). In this stage, the disease no longer responds well to disease-modifying drugs, and patients' disabilities steadily worsen. The destruction of neurons from early in the natural course of MS suggests that the progressive disabilities of MS might be the result of an accumulated neuronal loss that eventually overwhelms the brain's compensatory abilities. Primary progressive MS is a type of multiple sclerosis where there are no relapses, but over a period of years, there is gradual loss of physical and cognitive functions.

While the multiple sclerosis symptoms and course of illness can vary from person to person, there are three forms of the disease-relapsing-remitting MS, secondary progressive MS, and primary progressive MS.

All therapies currently approved for the treatment of MS act to reduce the number of attacks. These therapies are immunomodulators, and they do not act to fundamentally alter the course of MS, instead providing a moderate effect at reducing relapses and slowing disease progression as measured by the Expanded Disability Status Scale (EDSS). Studies show that current therapies reduce relapses by approximately 30-68% and reduce the percentage of relapsing MS patients who experience the relative risk of sustained disability within the range 30-42% (Havrdova et al. (2010) Neurology 74: (suppl 3), S3-S7). Further there were antibodies such as BIIB033 (Biogen Idec) in phase I clinical development, which aimed to provide primary neuroprotective/neuroregenerative efficacy. Preclinical data suggests that BIIB033 may act to repair axonal and/or myelin damage (Mi et al. (2007) Nature Medicine 13: 1228-1233).

Antagonists and inverse agonists of the Histamine H3 receptor have been disclosed in numerous patent applications. It has been identified that H3 receptors are predominantly expressed in the mammalian central nervous system (CNS), with minimal expression in peripheral tissues except on some sympathetic nerves (Leurs et al. (1998) Trends Pharmacol. Sci. 19: 177-183). Activation of H3 receptors by selective agonists or histamine results in the inhibition of neurotransmitter release from a variety of different nerve populations, including histaminergic and cholinergic neurons (Schlicker et al. (1994) Fundam. Clin. Pharmacol. 8: 128-137). Additionally, in vitro and in vivo studies have shown that H3 antagonists can facilitate neurotransmitter release in brain areas such as the cerebral cortex and hippocampus, relevant to cognition (Onodera et al. (1998) In: The Histamine H3 receptor, ed Leurs and Timmerman, pp 255-26′7, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al. (1999) Behav. Brain Res. 104: 147-155). Antagonists and partial agonists have been investigated for use in the treatment of cognitive impairment in neurological diseases.

### BRIEF DESCRIPTION OF THE INVENTION

H3 receptors have now been discovered on differentiating oligodendrocytes. In addition, compounds which act as H3 receptor inverse agonists have been shown to promote oligodendrocyte precursor differentiation. The inventors have also shown that inverse agonists of the H3 receptor can enhance remyelination through increased oligodendrocyte precursor differentiation. Thus, inverse agonists of the H3 receptor can be used as a novel therapy for treating MS and other demyelinating diseases.

In the first aspect of this invention, there is provided a compound which is an inverse agonist of the H3 receptor for use in the treatment of MS, where the treatment slows, halts or reverses the progression of disability. In another aspect of the invention, there is provided a compound which is an inverse agonist of the H3 receptor for use in slowing, halting or reversing the progression of disability in MS.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof.

The present invention provides 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in the treatment of MS in a human by oral administration at a dosage of from 5 to 500 micrograms per day. In one embodiment, the present invention provides 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in the treatment of MS in a human at a dosage of from 10 to 150 micrograms per day for oral administration. In another embodiment, the present invention provides 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in the treatment of MS in a human at a dosage of about 80 micrograms per day for oral administration.

In a second aspect of the invention, there is provided a compound which is an inverse agonist of the H3 receptor for use in promoting remyelination.

In one embodiment of the invention the H3 receptor inverse agonist is 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof.

In a third aspect of the invention, there is provided a compound which is an inverse agonist of the H3 receptor for use in the treatment of demyelinating diseases.

In one embodiment of the invention the H3 receptor inverse agonist is 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof.

In a fourth aspect, the present invention provides a pharmaceutical composition for oral administration to a human comprising a compound which is an inverse agonist of the H3 receptor, wherein the pharmaceutical composition is for use in the treatment of MS or demyelinating diseases. In one embodiment, the pharmaceutical composition comprises from 5 to 500 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in oral administration to a human per day. In one embodiment, the pharmaceutical composition comprises from 10 to 150 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in oral administration to a human per day. In one embodiment, the pharmaceutical composition comprises about 80 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in oral administration to a human per day.

### DESCRIPTION OF THE FIGURES

FIGS. 1a-1c. Effect of H3R example compounds in OPC differentiation assay.

FIG. 2. Effect of the Example compound 2, a H3R inverse agonist, on expression of mature oligodendrocyte markers in OPC differentiation assay.

FIG. 3a. Effect of the Example compound 1, a H3R inverse agonist, in OPC differentiation assay (n=2).

FIG. 3b. Effect of the Example compound 1 in OPC differentiation assay (n=5).

FIG. 4a. Effect of H3R knockdown via siRNA on OPC differentiation.

FIG. 4b. Effect of H3R knockdown via siRNA on OPC differentiation with statistical analysis.

FIG. 5a. Effect of Example compound 1, on remyelination in the cuprizone model (Forebrain, corpus callosum, Black-gold II staining).

FIG. 5b. Effect of Example compound 1, on remyelination in the cuprizone model (Hindbrain, corpus callosum, Black-gold II staining).

FIG. 5c. Statistical analysis of effect of Example compound 1 on remyelination in the cuprizone model (Corpus callosum, demyelination area).

FIG. 5d. Statistical analysis of effect of Example compound 1 on remyelination in the cuprizone model (Corpus callosum, mean intensity).

FIG. 5e. Effect of Example compound 2 on remyelination in cuprizone model (Forebrain, corpus callosum).

FIG. 5f. Effect of Example compound 2 on remyelination in cuprizone model (Forebrain, cortex).

FIG. 6a. Effect of Example compound 2 on intracellular level of cAMP in primary OPCs (n=1).

FIG. 6b. Effect of Example compound 2 on intracellular level of cAMP in primary OPCs (n=2).

FIG. 7. Effect of Example compound 1 on basal GTPγS binding to H3R.

### DETAILED DESCRIPTION OF THE INVENTION

In the first aspect of this invention, there is provided a compound which is an inverse agonist of the H3 receptor for use in the treatment of MS, where the treatment slows, halts or reverses the progression of disability. In another aspect of the invention, there is provided a compound which is an inverse agonist of the H3 receptor for use in slowing, halting or reversing the progression of disability in MS. In another aspect of the invention, there is provided a compound which is an inverse agonist of the H3 receptor for use in halting or reversing the progression of disability in MS. In another aspect of the invention, there is provided a compound which is an inverse agonist of the H3 receptor for use in slowing the progression of disability in MS.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

The present invention provides 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in the treatment of MS in a human by oral administration at a dosage of from 5 to 500 micrograms per day. In one embodiment, the present invention provides 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in the treatment of MS in a human at a dosage of from 10 to 150 micrograms per day for oral administration.

In another embodiment, the present invention provides 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in the treatment of MS in a human at a dosage of about 80 micrograms per day for oral administration.

There is also provided the use of a compound which is an inverse agonist of the H3 receptor for the manufacture of a medicament for the treatment of MS, where the treatment slows, halts or reverses the progression of disability. In another aspect of the invention, there is provided the use of a compound which is an inverse agonist of the H3 receptor for the manufacture of a medicament for use in slowing, halting or reversing the progression of disability in MS.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

There is also provided a method of treatment of MS, in mammals including humans, where the treatment slows, halts or reverses the progression of disability, which comprises administering to the sufferer a therapeutically effective amount of a compound which is an inverse agonist of the H3 receptor.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

The present invention provides a method of treatment of MS in humans, where the treatment slows, halts or reverses the progression of disability, which comprises administering orally to the human 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof at a dosage of from 5 to 500 micrograms per day. In one embodiment, the present invention provides a method of treatment of MS in humans, where the treatment slows, halts or reverses the progression of disability, which comprises administering orally to the human 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof at a dosage of from 10 to 150 micrograms per day. In another embodiment, the present invention provides a method of treatment of MS in humans, where the treatment slows, halts or reverses the progression of disability, which comprises administering orally to the human 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof at a dosage of about 80 micrograms per day.

In one embodiment of the invention, the treatment slows, halts or reverses the progression of disability by promoting differentiation of oligodendrocyte precursor cells.

In a second aspect of the invention, there is provided a compound which is an inverse agonist of the H3 receptor for use in promoting remyelination.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole, 4-(4-((1-isopropylpiperidin-4-yl)oxy)piperidin-1-yl)benzonitrile; or pharmaceutically acceptable salts thereof.

In one embodiment of the invention the H3 receptor inverse agonist is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

In another embodiment of the invention, the compound is 1-(3-(3-(4-chlorophenyl)propoxy)propyl)-piperidine, (R)-6-(4-(3-(2-methylpyrrolidin-1-yl)propoxy)phenyl)pyridazin-3 (2H)-one, or 3-(1H-imidazol-4-yl)propyl 4-chlorobenzylcarbamimidthioate dihydrobromide; or pharmaceutically acceptable salts thereof.

There is also provided the use of a compound which is an inverse agonist of the H3 receptor for the manufacture of a medicament for promoting remyelination.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

In another embodiment of the invention, the compound is 1-(3-(3-(4-chlorophenyl)propoxy)propyl)-piperidine, (R)-6-(4-(3-(2-methylpyrrolidin-1-yl)propoxy)phenyl)pyridazin-3(2H)-one, or 3-(1H-imidazol-4-yl)propyl 4-chlorobenzylcarbamimidthioate dihydrobromide; or pharmaceutically acceptable salts thereof.

There is also provided a method of promoting remyelination, in mammals including humans, which comprises administering to a subject in need thereof, a therapeutically effective amount of a compound which is an inverse agonist of the H3 receptor.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

In another embodiment of the invention, the compound is 1-(3-(3-(4-chlorophenyl)propoxy)propyl)-piperidine, (R)-6-(4-(3-(2-methylpyrrolidin-1-yl)propoxy)phenyl)pyridazin-3(2H)-one, or 3-(1H-imidazol-4-yl)propyl 4-chlorobenzylcarbamimidthioate dihydrobromide; or pharmaceutically acceptable salts thereof.

In a third aspect of the invention, there is provided a compound which is an inverse agonist of the H3 receptor for use in the treatment of demyelinating diseases.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole, 4-(4-((1-isopropylpiperidin-4-yl)oxy)piperidin-1-yl)benzonitrile; or pharmaceutically acceptable salts thereof.

In one embodiment of the invention the H3 receptor inverse agonist is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

In another embodiment of the invention, the compound is 1-(3-(3-(4-chlorophenyl)propoxy)propyl)-piperidine, (R)-6-(4-(3-(2-methylpyrrolidin-1-yl)propoxy)phenyl)pyridazin-3(2H)-one, or 3-(1H-imidazol-4-yl)propyl 4-chlorobenzylcarbamimidthioate dihydrobromide; or pharmaceutically acceptable salts thereof.

There is also provided the use of a compound which is an inverse agonist of the H3 receptor for the manufacture of a medicament for the treatment of demyelinating diseases.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

In another embodiment of the invention, the compound is 1-(3-(3-(4-chlorophenyl)propoxy)propyl)-piperidine, (R)-6-(4-(3-(2-methylpyrrolidin-1-yl)propoxy)phenyl)pyridazin-3(2H)-one, or 3-(1H-imidazol-4-yl)propyl 4-chlorobenzylcarbamimidthioate dihydrobromide; or pharmaceutically acceptable salts thereof.

There is also provided a method of treatment of demyelinating diseases, in mammals including humans, which comprises administering to a subject in need thereof, a therapeutically effective amount of a compound which is an inverse agonist of the H3 receptor.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

In one embodiment of the invention, the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

In a fourth aspect, the present invention provides a pharmaceutical composition for oral administration to a human comprising a compound which is an inverse agonist of the H3 receptor and one or more pharmaceutically acceptable excipients. In one embodiment, the pharmaceutical composition comprises from 5 to 500 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in oral administration to a human per day. In one embodiment, the pharmaceutical composition comprises from 10 to 150 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in oral administration to a human per day. In one embodiment, the pharmaceutical composition comprises about 80 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in oral administration to a human per day.

In a fifth aspect, the present invention provides a compound which is an inverse agonist of the H3 receptor for use in the treatment of MS. In one embodiment, the H3 receptor inverse agonist is 1-(3-(3-(4-chlorophenyl)propoxy)propyl)-piperidine, or a pharmaceutically acceptable salt thereof. In one embodiment, the H3 receptor inverse agonist is (R)-6-(4-(3-(2-methylpyrrolidin-1-yl)propoxy)phenyl)pyridazin-3(2H)-one, or a pharmaceutically acceptable salt thereof.

There is also provided the use of a compound which is an inverse agonist of the H3 receptor for the manufacture of a medicament for the treatment of MS. In one embodiment, the H3 receptor inverse agonist is 1-(3-(3-(4-chlorophenyl)propoxy)propyl)-piperidine, or a pharmaceutically acceptable salt thereof. In one embodiment, the H3 receptor inverse agonist is (R)-6-(4-(3-(2-methylpyrrolidin-1-yl)propoxy)phenyl)pyridazin-3(2H)-one, or a pharmaceutically acceptable salt thereof.

There is also provided a method of treatment of MS, in mammals including humans, which comprises administering to the sufferer a therapeutically effective amount of a compound which is an inverse agonist of the H3 receptor. In one embodiment, the H3 receptor inverse agonist is 1434344-chlorophenyl)propoxy)propyl)-piperidine, or a pharmaceutically acceptable salt thereof. In one embodiment, the H3 receptor inverse agonist is (R)-6-(4-(3-(2-methylpyrrolidin-1-yl)propoxy)phenyl)pyridazin-3(2H)-one, or a pharmaceutically acceptable salt thereof.

As used herein an inverse agonist of the H3 receptor is a compound that stabilizes the H3 receptor in an inactive conformation. This stabilization results in the decrease of spontaneous coupling of the receptor to G protein, thereby suppressing constitutive activity. In the absence of constitutive activity, inverse agonists behave as antagonists, therefore much of the previous literature describes compounds as H3 antagonists and/or inverse agonists. Inverse agonism of H3 receptors, and an assay thereof, has been reported (Constitutive Activity of Histamine H3 Receptors Stably Expressed in SK-N-MC Cells: Display of Agonism and Inverse Agonism by H3 Antagonists, Wieland et al. (2001) JPET 299:908-914).

An inverse agonists of the H3 receptor can also be functionally defined as a compound binding to the H3 receptor and at the same time increasing intracellular level of cAMP and activating the cAMP pathway.

Measurement of neurological impairment in individuals with multiple sclerosis is accomplished by using a scale that assesses various functional systems; the most widely used are The Expanded Disability Status Scale (EDSS) or the Multiple Sclerosis Functional Composite (MSFC). Scoring on the EDSS ranges from 0 (normal neurological examination) to 10.0 (death). The MSFC is made up of 3 components that measure arm and hand dexterity, walking speed and cognition. These scales have been used to assess both disability progression in individual patients and to measure the impact of current approved treatments in large scale clinical studies. The clinical studies treatment effect can be evaluate both as a mean change for the whole population or as the number of subjects with given change sustained over a given period of time.

None of the currently approved treatments have clearly demonstrated halting or reversal of disability progression, nor significant slowing of disability progression, measured by EDSS or MSFC.

Treatment with an H3 inverse agonist is expected to promote remyelination. Remyelination will have two possible outcomes: a) restoration of normal nerve impulse conduction (saltatory conduction), thereby potentially reversing disability/impairment; and b) protecting axons from acute or chronic degenerative mechanisms, thereby preventing axon loss and consequent disability progression. Disability can be measured using a functional scale such as EDSS and MSFC.

In another aspect of the invention, there is provided a compound which is 3-(benzo[d][1,3]dioxol-5-yl)-5-((1-cyclobutylpiperidin-4-yl)methyl)-1,2,4-oxadiazole, or a pharmaceutically acceptable salt thereof.

Synthetic Processes

1-{6-[(3-cyclobutyl-2,3,4,5-tetrahydro-1H-3-benzazepin-7-yl)oxy]-3-pyridinyl}-2-pyrrolidinone, and its pharmaceutically acceptable salts, can be produced by the processes referred to in WO2004/056369, or by the processes referred to in WO2005/123723.

4-(4-((1-isopropylpiperidin-4-yl)oxy)piperidin-1-yl)benzonitrile, and its pharmaceutically acceptable salts, can be produced as described in WO 2005/014571.

3-(benzo[d][1,3]dioxol-5-yl)-5-((1-cyclobutylpiperidin-4-yl)methyl)-1,2,4-oxadiazole can be produced according to Scheme 1 below:

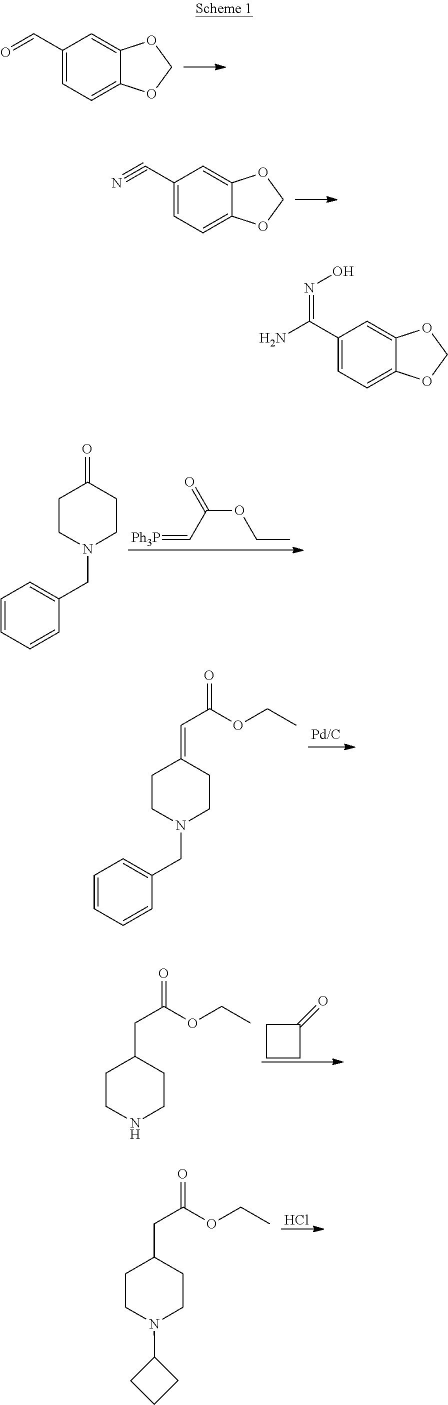

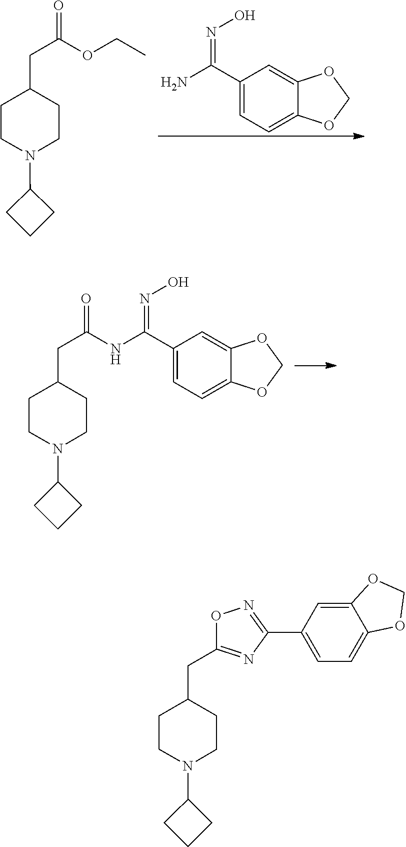

Because of their potential use in medicine, salts of the compounds of the invention are desirably pharmaceutically acceptable. For a review on suitable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent.

A pharmaceutically acceptable base addition salt can be formed by reaction of a compound of the invention with a suitable inorganic or organic base, (e.g. triethylamine, ethanolamine, triethanolamine, choline, arginine, lysine or histidine), optionally in a suitable solvent, to give the base addition salt which is usually isolated, for example, by crystallisation and filtration. Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

A pharmaceutically acceptable acid addition salt can be formed by reaction of a compound of the invention with a suitable inorganic or organic acid (such as hydrobromic, hydrochloric, sulphuric, nitric, phosphoric, succinc, maleic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of the invention can comprise or be for example a hydrobromide, hydrochloride, sulfate, nitrate, phosphate, succinate, maleate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

Other non-pharmaceutically acceptable salts, e.g. formates, oxalates or trifluoroacetates, may be used, for example in the isolation of the compounds of the invention and are included within the scope of this invention.

Pharmaceutically acceptable salts of 1-{6-[(3-cyclobutyl-2,3,4,5-tetrahydro-1H-3-benzazepin-7-yl)oxy]-3-pyridinyl}-2-pyrrolidinone include those described in WO2004/056369. Pharmaceutically acceptable salts of 4-(4-((1-isopropylpiperidin-4-yl)oxy)piperidin-1-yl)benzonitrile include those described in WO 2005/014571. Pharmaceutically acceptable salts of 3-(benzo[d][1,3]dioxol-5-yl)-5-((1-cyclobutylpiperidin-4-yl)methyl)-1,2,4-oxadiazole can be produced as described above, in particular the acid addition salts as described above, and in particular include the hydrochloride salt.

Uses

Inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts, can be used in slowing, halting or reversing the progression of disability in MS. In one aspect, inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts, can be used in promoting remyelination by restoration of normal nerve impulse conduction (saltatory conduction), thereby potentially reversing disability/impairment. In another aspect, inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts, can be used in promoting remyelination by protecting axons from acute or chronic degenerative mechanisms, thereby preventing axon loss and consequent disability progression.

Inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts, can be used in the treatment of Multiple Sclerosis including Radiological Isolated Syndrome, Clinical Isolated Syndrome, Relapsing-Remitting multiple sclerosis, secondary progressive multiple sclerosis, primary progressive multiple sclerosis, progressive/relapsing multiple sclerosis, neuromyelitis optica, and Acute MS (Marburg's variant). In one aspect, inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts, can be used in the treatment of Relapsing-Remitting multiple sclerosis (RRMS). In another aspect, inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts, can be used in the treatment of secondary progressive multiple sclerosis (SPMS).

Inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts thereof, also can be used in the treatment of diseases causing demylination, for example, Acute Disseminated encephalomyelitis, Optic neuritis, Vitamin B12 deficiency, Central Pontine myelinolysis, Tabes Dorsalis, Transverse myelitis, Progressive Multifocal leukoencephalopathy and Leukodystrophies.

Inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts, also can be used in the treatment of dementing diseases with an aspect of demylination including vascular dementia, mixed dementia and Alzheimer's disease.

Inverse agonists of the H3 receptor and their pharmaceutically acceptable salts thereof, particularly 1-{6-[(3-cyclobutyl-2,3,4,5-tetrahydro-1H-3-benzazepin-7-yl)oxy]-3-pyridinyl}-2-pyrrolidinone and its pharmaceutically acceptable salts thereof, also have potential use in the treatment of demyelinating diseases of the peripheral nervous system including Chronic inflammatory demyelinating polyneuropathy, Guillain-Barré syndrome, Acute inflammatory demyelinating polyneuropathy, Miller Fisher syndrome, Acute motor axonal neuropathy, Acute motor sensory axonal neuropathy, Acute panautonomic neuropathy. Bickerstaff's brainstem encephalitis, Anti-MAG peripheral neuropathy, Charcot-Marie-Tooth Disease and Copper deficiency.

Dosage

The compounds of the present invention can be administered orally. A compound of the invention can be administered to a human at a dosage of from about 1 to about 1000 micrograms per day, or from about 5 to about 500 micrograms per day, or from 10 to 150 micrograms per day.

The present invention provides 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof for use in the treatment of MS or demyelinating diseases in a human by oral administration a dosage of from 5 to 500 micrograms per day of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof. In one embodiment, 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof is provided at a dosage of from 10 to 150 micrograms per day for oral administration in a human. In one embodiment, 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof is provided at a dosage of about 80 micrograms per day for oral administration in a human.

Pharmaceutical Compositions

Compounds of the invention may be formulated as a pharmaceutical dosage form, containing the compound of the invention or a pharmaceutically acceptable salt thereof, and one or more pharmaceutically acceptable excipients. Such dosage forms and excipients are described in the art. For example, 1-{6-[(3-cyclobutyl-2,3,4,5-tetrahydro-1H-3-benzazepin-7-yl)oxy]-3-pyridinyl}-2-pyrrolidinone can be formulated in pharmaceutical compositions, and at dosage levels, as described in WO2004/05369 and WO2008/104590.

The present invention provides a pharmaceutical composition for oral administration to a human comprising a compound which is an inverse agonist of the H3 receptor and one or more pharmaceutically acceptable excipients, wherein the pharmaceutical composition is for use in the treatment of MS or demyelinating diseases. In one embodiment, the pharmaceutical composition comprises 5 to 500 micrograms of the compound of the invention or a pharmaceutically acceptable salt thereof for use in oral administration to a human per day. In one embodiment, the pharmaceutical composition comprises 10 to 150 micrograms of the compound of the invention or a pharmaceutically acceptable salt thereof for use in oral administration to a human per day. In one embodiment, the pharmaceutical composition comprises about 80 micrograms of the compound of the invention or a pharmaceutically acceptable salt thereof for use in oral administration to a human per day.

Combinations

Compounds administered according to the invention may be used in combination with other therapeutic agents, for example medicaments claimed to be useful in the treatment of Multiple Sclerosis. Suitable examples of such other therapeutic agents may be glatiramer acetate (Copaxone), β interferon-1a (Avonex and Rebif), β interferon-1b (Betaseron), fingolimod (Gilenya), and natalizumab (Tysabri). Other suitable agents include BG-12, Peg-Avonex, laquinimod, teriflunomide, daclizumab (Zenapax), alemtuzumab (Campath), BAF312, ONO-4641, ponesimod, Pleneva, plovamer, ATX-MS-1467, Trimesta, V85546, ATL-1102, ofatumumab, secukinumab, LY-2127399, toxavin, manocort and Firategrast.

When the compound of the invention or a pharmaceutically acceptable salt thereof is used in combination with other therapeutic agents, the compounds may be administered either sequentially or simultaneously by any convenient route.

The invention thus provides, in a further aspect, a combination 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When the compound of the invention or a pharmaceutically acceptable salt thereof is used in combination with a second therapeutic agent active against the same disease state the dose of each compound may differ from that when the compound is used alone.

### EXAMPLES

### Abbreviations

bFGF basic fibroblast growth factor

BDM basal chemically defined medium

BSA bovine serum albumin

CNS central nervous system

CPZ cuprizone

EDSS Expanded Disability Status Scale

GPCR G protein coupled receptor

H3R histamine receptor 3

IOD Integrated Optical Density

MAG myelin-associated glycoprotein

MBP myelin basic protein

MS Multiple Sclerosis

NAc N-acetyl-L-cystenine

OCT optimal cutting temperature solution

OPC oligodendrocyte precursor cell

PBS Phosphate buffered saline

PDGF platelet-derived growth factor

PDL poly-D-lysine

PFA paraformaldehyde

PO poly-ornithine

RRMS relapsing-remitting MS

RT-PCR polymerase chain reaction

SEM standard error of the mean

siRNA small interfering RNA

SPMS secondary progressive MS

### Description 1: Benzo[d][1,3]dioxole-5-carbonitrile

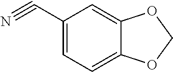 [SMILES: N#Cc1ccc2c(c1)OCO2]

To a solution of benzo[d][1,3]dioxole-5-carbaldehyde (16 g, 107 mmol), sodium formate (14.50 g, 213 mmol) in Formic Acid (150 mL) was added solid hydroxylamine hydrochloride (22.22 g, 320 mmol). The reaction mixture was stirred at 100° C. for 2 hr. The solvent was removed out under reduced pressure. Then H₂O (200 mL) was added in, the mixture was extracted with DCM (200 mL×3), the combined organic layer was dried over sodium sulfate and evaporated under reduced pressure to afford benzo[d][1,3]dioxole-5-carbonitrile as a yellow solid (15 g, 91%).

¹H NMR (400 MHz, CDCl₃) δ: 7. 21 (d, T=8.0 Hz, 1H), 7.03 (s, 1H), 6.86 (d, T=8.0 Hz, 1H), 6.07 (s, 2H).

### Description 2: (Z)—N′-hydroxybenzo[d][1,3]dioxole-5-car boximidamide

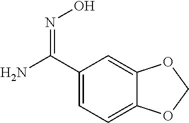 [SMILES: N/C(=N/O)c1ccc2c(c1)OCO2]

To a solution of benzo[d][1,3]dioxole-5-carbonitrile (15 g, 102 mmol), hydroxylamine hydrochloride (14.17 g, 204 mmol) in Ethanol (500 mL) was added Na₂CO₃ (54.0 g, 510 mmol) in one charge. The reaction mixture was stirred at 80° C. for 5 hr. Then the solvent was removed under reduced pressure, the residual was washed with DCM (1 L×4), filtered and the combined filtrate was concentrated under reduced pressure to afford (Z)—N′-hydroxy benzo[d][1,3]dioxole-5-carboximidamide as a yellow solid (15 g, 73.5%). MS (ES⁺) m/z 181.1 (MH⁺).

### Description 3: Ethyl 2-(1-benzylpiperidin-4-ylidene)acetate

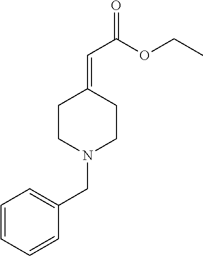 [SMILES: CCOC(=O)C=C1CCN(Cc2ccccc2)CC1]

To a solution of 1-benzylpiperidin-4-one (30 g, 159 mmol) in N,N-Dimethylformamide (DMF) (400 mL) was added solid ethyl 2-(triphenylphosphoranylidene)acetate (110 g, 317 mmol). The reaction mixture was stirred at 120° C. for 20 hr. The reaction mixture was filtered and the filtrate was concentrated under reduced pressure, the crude product was purified by column chromatography on silica gel (Petroleum ether:Ethyl Acetate=20:1) to afford ethyl 2-(1-benzylpiperidin-4-ylidene)acetate as a yellow oil (24 g, 55.5%). MS (ES⁺) m/z 259.9 (MH⁺).

### Description 4: Ethyl 2-(piperidin-4-yl)acetate

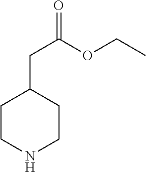

To a solution of ethyl 2-(1-benzylpiperidin-4-ylidene)acetate (24 g, 93 mmol) in Ethyl acetate (300 mL) was added Pd/C (3.94 g). The mixture was hydrogenated using the H-cube (settings: 55° C., 55 psi) for 16 hr. The reaction mixture was filtered and the filtrate was concentrated under reduced pressure to afford ethyl 2-(piperidin-4-yl)acetate as a colorless oil (15 g, 90%).

¹H NMR (400 MHz, CDCl₃) δ: 4.10 (m, 2H), 3.03 (d, J=16.4 Hz, 2H), 2.59 (t, J=16.4 Hz, 2H), 2.19 (d, J=9.6 Hz, 2H), 1.87 (m, 1H), 1.67 (d, J=17.2 Hz, 2H), 1.23 (t, J=9.6 Hz, 3H), 1.10 (m, 2H).

### Description 5: Ethyl 2-(1-cyclobutylpiperidin-4-yl)acetate

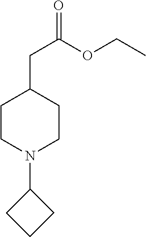 [SMILES: CCOC(=O)CC1CCN(C2CCC2)CC1]

To a solution of ethyl 2-(piperidin-4-yl)acetate (13 g, 76 mmol), cyclobutanone (6.92 g, 99 mmol) in Dichloromethane (DCM) (200 mL) stirred in air at 20° C. was added acetic acid (4.35 mL, 76 mmol) in one charge. The reaction mixture was stirred at 25° C. for 0.5 hr. Then sodium triacetoxyborohydride (25.7 g, 121 mmol) was added in the mixture above. The reaction mixture was stirred at 25° C. for 3 hr. The mixture was washed with NaOH solution (2M, 150 mL), the inorganic layer was extracted with DCM (200 mL×3), the combined organic layer was dried over sodium sulfate and evaporated under reduced pressure to afford ethyl 2-(1-cyclobutylpiperidin-4-yl)acetate as a colourless oil (15 g, 79%).

¹H NMR (400 MHz, CDCl₃) δ: 4.06 (m, 2H), 3.23 (d, J=15.6 Hz, 2H), 3.01 (m, 1H), 1.96-2.28 (m, 8H), 1.70-1.81 (m, 4H), 1.48-1.65 (m, 3H), 1.19 (t, J=9.6 Hz, 3H).

### Description 6: Preparation of 2-(1-cyclobutylpiperidin-4-yl)acetic acid

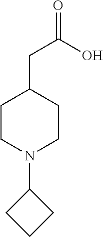 [SMILES: O=C(O)CC1CCN(C2CCC2)CC1]

The mixture of ethyl 2-(1-cyclobutylpiperidin-4-yl)acetate (18 g, 80 mmol) and HCl. (12M, 150 mL) was heated to 100° C. and the reaction mixture was stirred at 100° C. for 5 hr. Then the solvent was removed under reduced pressure to afford 2-(1-cyclobutylpiperidin-4-yl)acetic acid as a brown solid (12 g, 68.5%).

¹H NMR (400 MHz, DMSO) δ: 3.51 (m, 1H), 3.26 (d, J=12.0 Hz, 2H), 2.67 (m, 2H), 2.33 (m, 2H), 2.17 (d, J=6.8 Hz, 2H), 2.12 (m, 2H), 1.82 (m, 3H), 1.68 (m, 2H), 1.51 (m, 2H).

### Description 7: (Z)—N-(benzo[d][1,3]dioxol-5-yl(hydroxyimino)methyl)-2-(1-cyclobutyl-piperidin-4-yl)acetamide

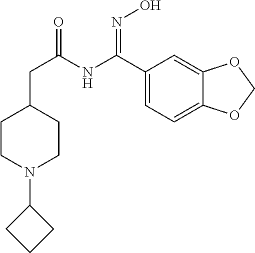 [SMILES: O=C(CC1CCN(C2CCC2)CC1)N/C(=N/O)c1ccc2c(c1)OCO2]

To a solution of 2-(1-cyclobutylpiperidin-4-yl)acetic acid (3 g, 15.21 mmol), HATU (8.67 g, 22.81 mmol) and DIPEA (7.97 mL, 45.6 mmol) in N,N-Dimethylformamide (DMF) (20 mL) stirred at 20° C. was added solid (Z)—N′-hydroxybenzo[d][1,3]dioxole-5-carboximidamide (2.74 g, 15.21 mmol). The reaction mixture was stirred at 20° C. for 1 hr. Then H₂O (50 mL) was added in, the mixture was extracted with DCM (30 mL×3), the combined organic layer was dried over sodium sulfate and evaporated under reduced pressure, the crude product was for the next step straightly (3 g, 49.4%). MS (ES⁺) m/z 360.2 (MH⁺).

### Example 1

### 1-{6-[(3-cyclobutyl-2,3,4,5-tetrahydro-1H-3-benzazepin-7-yl)oxy]-3-pyridinyl}-2-pyrrolidinone

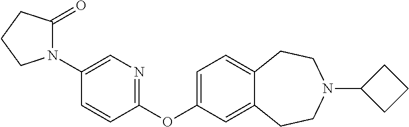 [SMILES: O=C1CCCN1c1ccc(Oc2ccc3c(c2)CCN(C2CCC2)CC3)nc1]

A mixture of cyclobutanone (3.77 kg) and acetic acid (1.074 kg) were added to a solution of 1-[6-(2,3,4,5-Tetrahydro-1H-3-benzazepin-7-yloxy)-3-pyridinyl]-2-pyrrolidinone (WO2005/123723A1, description 3) (5.8 kg) in dichloromethane (58 L) and the mixture stirred at 25-35° C. for 3 hours, then cooled to 10-15° C. Sodium triacetoxyborohydride (5.72 kg) was added in four equal portions at intervals of 10 minutes, maintaining the temperature at 10-15° C. The resulting mixture was heated to 25-35° C. and stirred for 3 hours until complete reaction, as determined by TLC.

The reaction mixture was cooled to 5-10° C., the pH adjusted to pH 10-11 with aqueous sodium hydroxide solution (7.4% w/w), stirred for 30-40 minutes and the phases separated. The aqueous phase was extracted with dichloromethane (3×17.5 L). The combined organic phases were washed twice with aqueous sodium chloride solution (13% w/w), filtered and the filtrate concentrated to less than 14.5 L. Methanol (29 L) was added, the mixture heated under reflux for 25-30 minutes and then concentrated under vacuum, maintaining the temperature below 50° C., to less than 14.5 L. The methanol addition, heating and concentration was repeated three times using 29 L methanol each time.

The residue was dissolved in dichloromethane (58 L), washed three times with aqueous sodium chloride solution (4.8%), filtered, and concentrated to less than 14.5 L. Methanol (29 L) was added, the mixture heated under reflux for 25-30 minutes and then concentrated under vacuum, maintaining the temperature below 50° C., to less than 14.5 L. The methanol addition, heating and concentration was repeated and the distillation continued to less than 8.7 L. The resulting slurry was dissolved in 2-propanol at reflux and distilled under vacuum, maintaining the temperature below 60° C., to less than 8.7 L. This operation was repeated using 29 L of 2-propanol. The residue was heated in 2-propanol (26 L) under reflux for 20 minutes, cooled 70-75° C. and a slurry of 1-{6-[(3-Cyclobutyl-2,3,4,5-tetrahydro-1H-3-benzazepin-7-yl)oxy]-3-pyridinyl}-2-pyrrolidinone (6 g) in 2-propanol (17 mL) added. The mixture was cooled to 25-35° C. over 50 minutes, then cooled to −5-0° C. and stirred for 3 hours. The slurry was filtered, the cake washed with cold 2-propanol (6 L) and dried under vacuum at 50-60° C. to give the title product (4.74 kg).

### Example 2

### 3-(benzo[d][1,3]dioxol-5-yl)-5-((1-cyclobutylpiperidin-4-yl)methyl)-1,2,4-oxadiazole

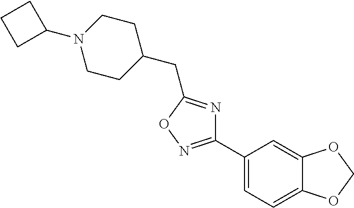 [SMILES: c1cc2c(cc1-c1noc(CC3CCN(C4CCC4)CC3)n1)OCO2]

A solution of (Z)—N-(benzo[d][1,3]dioxol-5-yl(hydroxyimino)methyl)-2-(1-cyclobutyl-piperidin-4-yl)acetamide (5 g, 13.91 mmol) in N,N-Dimethylformamide (DMF) (30 mL) was heated to 120° C. and the reaction mixture was stirred at 120° C. for 24 hr. The reaction mixture was filtered and the filtrate was concentrated under reduced pressure, the crude product was purified by Pre-HPLC to afford 3-(benzo[d][1,3]dioxol-5-yl)-5-((1-cyclobutylpiperidin-4-yl)methyl)-1,2,4-oxadiazole as a white solid (1.5 g, 31.6%).

¹H NMR (400 MHz, MeOD) δ: 7.60 (d, J=8.0 Hz, 1H), 7.44 (s, 1H), 6.94 (d, J=8.0 Hz, 1H), 6.04 (s, 2H), 2.90 (m, 4H), 2.74 (m, 1H), 2.04 (m, 2H), 1.68-2.03 (m, 10H), 1.40 (m, 2H).

MS (ES⁺) m/z 342.1 (MH⁺)

### Example 3

### 4-(4-((1-isopropylpiperidin-4-yl)oxy)piperidin-1-yl)benzonitrile hydrochloride

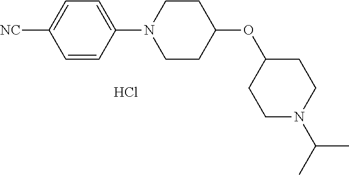 [SMILES: CC(C)N1CCC(OC2CCN(c3ccc(C#N)cc3)CC2)CC1 ; Cl]

4-(4-((1-isopropylpiperidin-4-yl)oxy)piperidin-1-yl)benzonitrile hydrochloride can be produced as described in WO2005014571.

### Comparative Example 4

### 4-[3-(Phenylmethoxy)propyl]-1H-imidazole oxalate

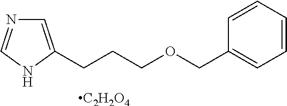

4-[3-(Phenylmethoxy)propyl]-1H-imidazole oxalate was purchased from a commercial source (Santa Cruz Biotechnology, USA).

### Comparative Example 5

### 4-[3-(1H-Imidazol-4-yl)propyl]piperidine dihydrobromide

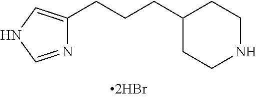 [SMILES: c1nc(CCCC2CCNCC2)c[nH]1]

4-[3-(1H-Imidazol-4-yl)propyl]piperidine dihydrobromide was purchased from a commercial source (Tocris Bioscience, UK).

### Example 6

### 1-(3-(3-(4-chlorophenyl)propoxy)propyl)piperidine

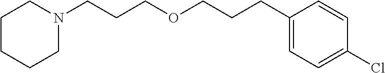 [SMILES: Clc1ccc(CCCOCCCN2CCCCC2)cc1]

1-(3-(3-(4-chlorophenyl)propoxy)propyl)piperidine, an inverse agonist of H3R (Schwartz, (2011) Br. J. Pharmacol. 163:713-721), was purchased from a commercial source (Tocris Bioscience, UK). Example 6 can be synthesized as described in U.S. Pat. No. 7,169,928.

### Example 7

### (R)-6-(4-(3-(2-methylpyrrolidin-1-yl)propoxy)phenyl)pyridazin-3(2H)-one

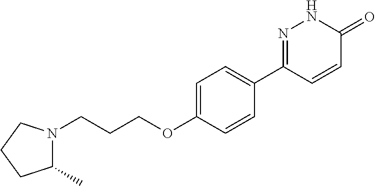 [SMILES: C[C@@H]1CCCN1CCCOc1ccc(-c2ccc(=O)[nH]n2)cc1]

(R)-6-(4-(3-(2-methylpyrrolidin-1-yl)propoxy)phenyl)pyridazin-3(2H)-one, an inverse agonist of H3R, can be synthesized as described in Hudkins et al. (J. Med. Chem. (2011) 54: 4781-4792) or in U.S. Pat. No. 8,207,168.

### Example 8

### 3-(1H-imidazol-4-yl)propyl 4-chlorobenzylcarbamimidothioate dihydrobromide

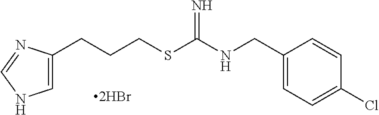 [SMILES: N=C(NCc1ccc(Cl)cc1)SCCCc1c[nH]cn1]

3-(1H-imidazol-4-yl)propyl 4-chlorobenzylcarbamimidothioate dihydrobromide (also known as clobenpropit) is an inverse agonist of H3R (Moreno-Delgado et al., Neuropharmacology (2006) 51: 517-523). It was purchased from a commercial source (Tocris Bioscience, UK).

### Biological Assays

### Example 9

### Oligodendrocyte Precursor Cell (OPC) Differentiation Assay

The effects of small-molecule inverse agonists and neutral antagonists of histamine receptor 3 (H3R) on OPC differentiation were determined using the following assay.

Cell Isolation

The enriched OPC were obtained from dissecting 2-d-old rat pups forebrain. The pups were anesthetized and decapitated. Then the skull was cut with microdissecting scissors and the cortex was taken out with Dumont forceps. The meninges were removed with fine tips. After repeated trituration of the cortex through cell strainer, cells were re-suspended with the culture media consisted of DMEM supplemented with 20% Fetal bovine serum (Invitrogen, Carlsbad, Calif.), glutamine (4 mM, (Invitrogen), and plated onto poly-D-lysine (PDL, 100 m/ml for 1 h, Sigma, St. Louis, Mo.)-coated T75 flasks. The resulting cultures were fed with fresh culture media twice per week and almost all the neurons will die due to the high concentration of serum in one week. Then the mixed glial cells were subjected to a series shake-off procedure to obtain pure OPCs two weeks later. Specifically, the mixed glial cells were shaken initially for 1 hour at 100 rpm to remove microglia and shaken for 20-22 hours at 37° C. at 200 rpm to enrich OPCs. OPCs were collected by centrifugation at 1200 rpm for 5 min, re-suspended in basal chemically defined medium (BDM). BDM consisted of DMEM supplemented with N2 medium (100×, Invitrogen), glutamine (4 mM, Invitrogen), BSA (0.1 mg/ml, Sigma), hydrocortisone (20 nM, Sigma), selenium (30 nM, Sigma) and biotin (10 nM, Sigma). OPCs were plated on poly-ornithine (PO, 50 μg/ml for 1 hr, Sigma)-coated culture dishes and maintained in BDM supplemented with bFGF (10 ng/ml, Invitrogen) and PDGF (10 ng/ml, PeproTech, Rocky Hill, N.J.) before experimental manipulation. OPCs were 95% pure judged by A2B5+ staining.

Drugs and Reagents

The antibodies used for immunoblot and immunocytochemistry experiments were rabbit anti-myelin basic protein (MBP, Millipore, Billerica, Mass.), mouse anti-myelin-associated glycoprotein (MAG, abcam, Cambridge, Mass.).

OPC Differentiation Assay

To initiate OPC differentiation, OPCs were firstly treated with 0.05% Trypsin/EDTA, and seeded into PO-coated 384-mini well plate at a density of 2,000 to 5,000 cells/well. Each test compound was dissolved in DMSO at the concentration of 10 mM for the assay. Compounds at different concentrations (full dose curve, 0.3 nM-10 uM) were added into the OPC culture in duplicates with Echo machine (Thermo, Waltham, Mass.). OPCs were cultured in BDM supplemented with N-acetyl-L-cystenine (30 μM, Sigma) to differentiate into mature oligodendrocytes for 4 days. BDM comprise DMEM (Invitrogen), N2 (100×, Invitrogen), L-Glu (50×, Invitrogen), Sodium Pyruvate (100×, Invitrogen), BSA (0.1%, Sigma), Biotin (10 nM, Sigma), hydrocortisone (10 nM, Sigma). The resulting cultures were fixed with 4% polyformaldehyde (Sigma) and samples were blocked with the blocking buffer (Donkey Serum 3%, Sigma; Triton X-100 0.04%, Sigma). Then fixed cells were incubated in primary antibodies (anti-myelin basic protein, MBP antibody, 600× dilution in blocking buffer) overnight at 4° C., washed thoroughly with PBS and then incubated with Alexa 488-labeled secondary antibody (Molecular Probes, Invitrogen) for 2 h at R.T; then washed thoroughly with PBS and stained with Dapi (1 ug/ml, Sigma),

Data Acquisition and Analysis

The full-plate immunocytochemistry staining was read by an automatic image-based analysis system-Cellomics (Thermo) and the raw data for each well was acquired as percentage of MBP+ population as follows: Pictures of eight fields per well were captured and analyzed quantitatively; the well data is the average of 8 fields. The intensity of anti-MBP staining in the cytosol was used as the criteria of positive objects. The threshold of positive objects was set based on the average intensity of the positive control well, for which triiodothyronine (15 nM) was used. Then the data was analyzed and presented as fold change of the number of the vehicle control well. Graphs were generated by softwares: Microsoft Excel and IDBS XL fits. The curve fitting was performed with software XL fits to derive EC50 of the positive compounds.

Western Blot

After treatments, OPCs were washed twice with ice-cold PBS and total cell lysates were harvested in RIPA lysis buffer (50 mM Tris-HCl, pH 7.5, 1 mM EDTA, 1 mM EGTA, 1 mM sodium orthovanadate, 50 mM sodium fluoride, 0.1% 2-mercaptoethanol, 1% triton X-100 and proteases inhibitor cocktail). The lysates were briefly sonicated and stored at −80° C. before Western analysis. The BCA Protein Assay Reagent (PIERCE, Thermo) was performed to determine protein concentration. 15 μg of total protein per sample was aliquoted, boiled for 5 min after being mixed with 2×SDS buffer (125 mM Tris-HCl, 4% SDS, 20% glycerol, 100 mM DTT) and separated by SDS-PAGE on Bis-Tris mini-gels (Invitrogen). Separated proteins were then transferred to nitrocellulose membranes and blocked in 5% milk/TBS-0.1% Tween for 1 h at room temperature. Membranes were then incubated in the presence of primary antibodies diluted in 5% milk/TBS-0.1% Tween overnight at 4° C. The following day, membranes were washed 3 times for 5 min with TBS-0.1% Tween and incubated for 1 h at room temperature in 5% milk/TBS-0.1%.

Tween containing goat anti-rabbit or goat anti-mouse secondary antibodies (Jackson ImmunoResearch Laboratories, West Grove, Pa.) at a dilution of 1:5000. The detection of HRP conjugated secondary antibodies was performed by enhanced chemiluminescence substrate mixture (PIERCE) using the FUJI imaging device (FUJI, Tokyo, Japan).

Statistical Analysis

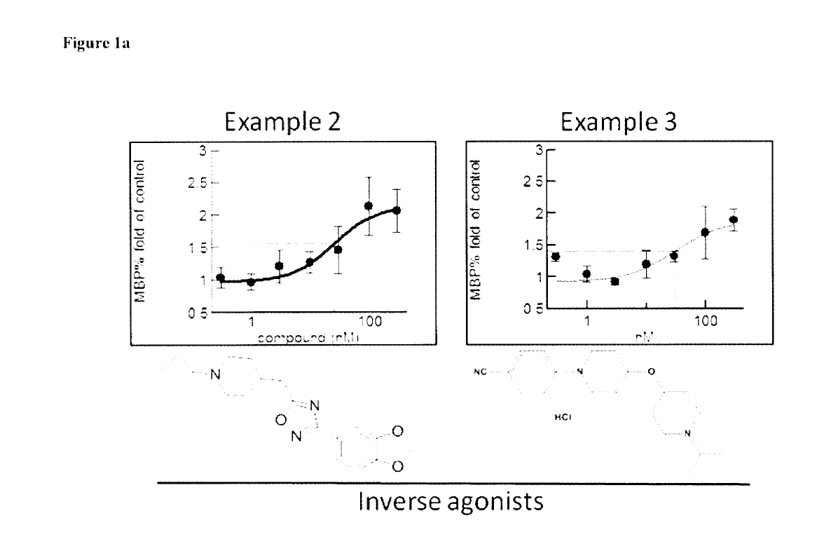

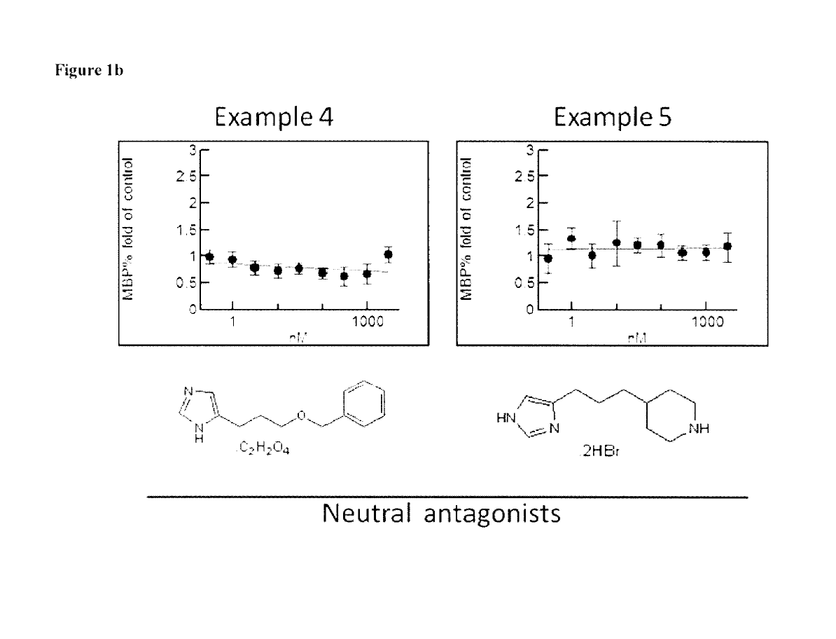

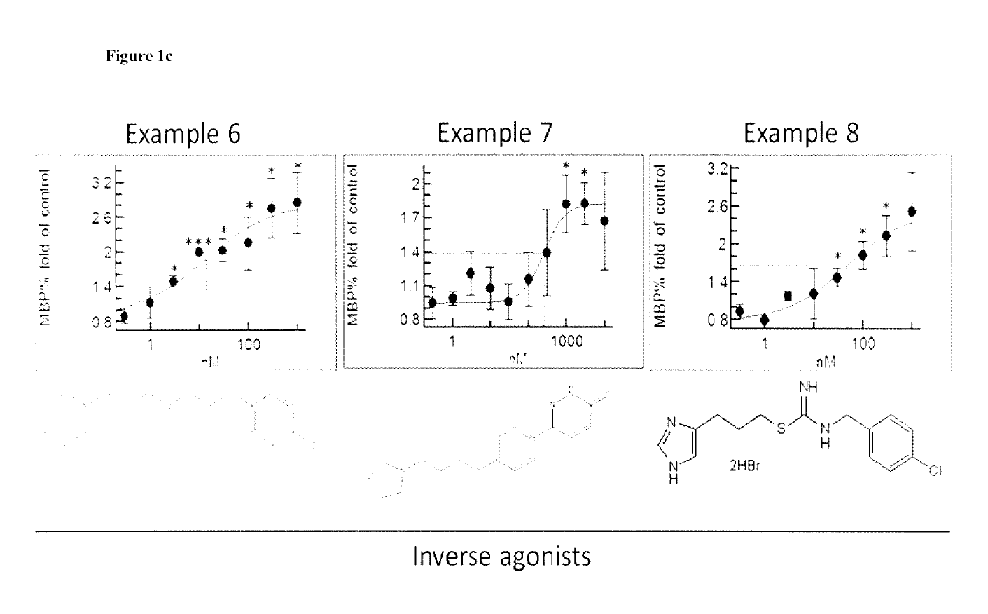

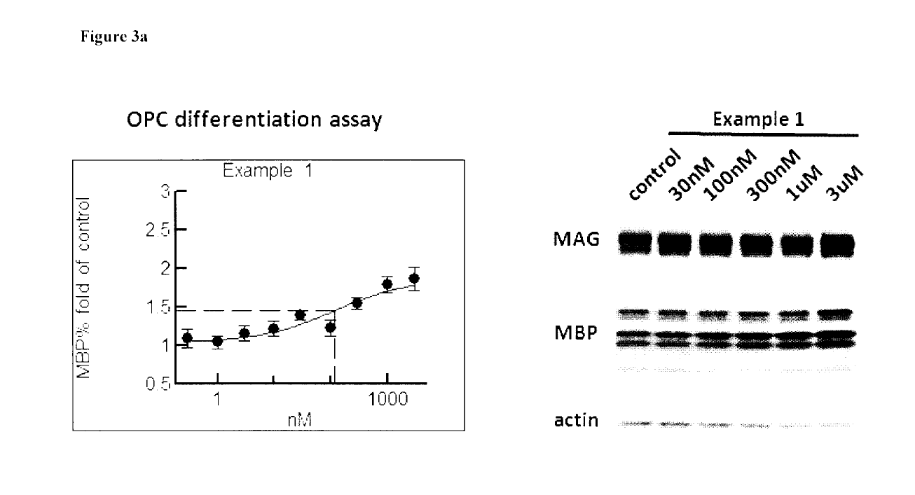

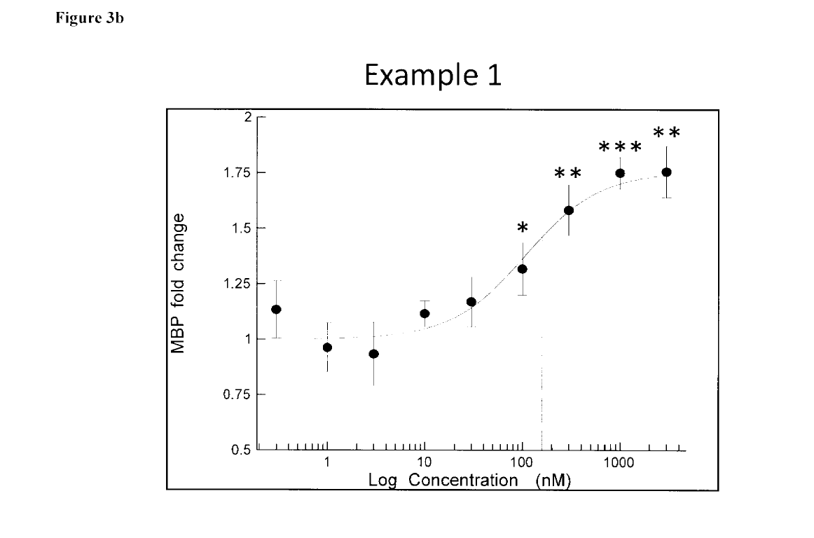

All data except FIG. 1 are given as mean±standard error of the mean (SEM). Data in FIG. 1 are presented as mean±standard deviation (SD). Statistical analysis in FIG. 3b was performed using one-way ANOVA or student's t test when appropriate. Statistical analysis in FIG. 1c was performed using student's t test. Statistical significance was inferred if p<0.05.

Results

As illustrated by FIG. 1, example compounds targeting H3R with diverse structures and profiles were tested in the OPC differentiation assay at a series of doses (0.3 nM-10 uM). Treatment with Example compound 2, which is a H3R inverse agonist, promoted OPC differentiation in a dose-dependent manner, at EC50=25 nM, as evident by increased MBP+ population shown in FIG. 1a. Similarly, treatment with another H3R inverse agonist, Example compound 3, also showed similar promotive effect, at EC50 of 31 nM. Treatment with Example compounds 6, 7 and 8, which are also H3R inverse agonists and are structurally different, showed similar promotive effect on oligodendrocyte differentiation, at EC50 of 14 nM, 250 nM, 48 nM, respectively, as shown in FIG. 1c. In contrast, treatment with neutral antagonists Example compound 4 and 5 didn't show any effect on OPC differentiation at all concentrations (FIG. 1b).

### Conclusion

Neutral antagonists of H3R can only inhibit histamine-dependent activity of the receptor; on the other hand, inverse agonists can inhibit both histamine-dependent activity and histamine-independent constitutive activity. Only inverse agonists showed positive effects in OPC differentiation assay, these results demonstrate that constitutive activity of H3R plays a critical role in regulating oligodendrocyte differentiation.

Results

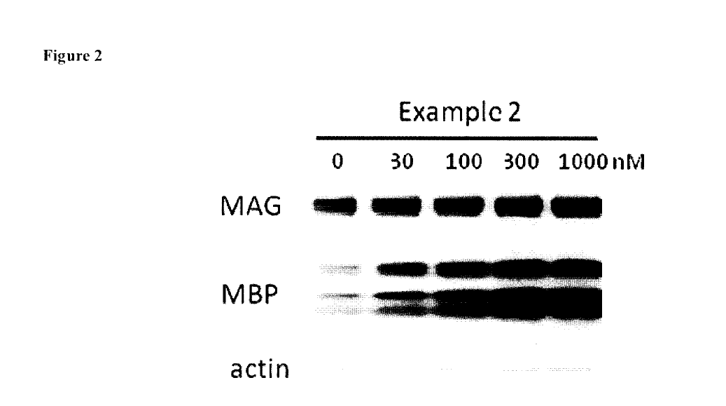

The compound of Example 2 was chosen for further analysis with western blot, as illustrated by FIG. 2. Consistently, western blot revealed a significant increase in expression levels of two markers of mature oligodendrocytes, myelin-associated glycoprotein (MAG) and myeline basic protein (MBP) in differentiating oligodendrocytes after treatment with Example compound 2, which suggests that treatment with Example compound 2 drives more OPCs to differentiate (FIG. 2).

Example compound 1, another H3R inverse agonist, demonstrated similar profile in the OPC differentiation assay as other inverse agonists. Treatment with Example compound 1 promoted OPC differentiation at EC50 of 118±49 nM (data from two experiments (n=2) are shown in FIG. 3a), as shown by more MBP+ cells and increased expression levels of MAG and MBP, two biomarkers for mature oligodendrocytes. The experiments were run three more times and the data from the five experiments (n=5) were subjected to statistic analysis. Analysis of variance (ANOVA) was used to compare the difference among the 10 dose groups (vehicle and 9 active doses). The p-value of ANOVA<0.0001 indicated a statistically significant difference. Data from the five experiments were averaged and expressed as fold change of the percentage in the vehicle control well. Treated vs vehicle, *, p<0.05, **, p<0.01, ***, p<0.001. As shown in FIG. 3b, Example compound 1 promoted OPC differentiation in vitro in a concentration-dependent manner with an EC₅₀ value of 159±57 nM, as shown by more MBP-positive cells (up to 1.8±0.1 folds of control, at 3 uM).

### Conclusion

Results of western blot were consistent with the result of quantitative immunocytochemistry analysis. Both Example compound 1 and Example compound 2 increased expression levels of mature oligodendrocyte markers in a dose-dependent manner

### Example 10

### H3R Knockdown Experiment

H3R specific small interfering RNA (siRNA) was used to knock down H3R expression in OPCs, and the resultant phenotype was investigated in the differentiation condition.

Cell Isolation

The enriched OPC were obtained from dissecting 2-d-old rat pups forebrain. The pups were anesthetized and decapitated. Then the skull was cut with microdissecting scissors and the cortex was taken out with Dumont forceps. The meninges were removed with fine tips. After repeated trituration of the cortex through cell strainer, cells were re-suspended with the culture media consisted of DMEM supplemented with 20% Fetal bovine serum, glutamine (4 mM), and plated onto poly-D-lysine (PDL, 100 m/ml for 1 h)-coated T75 flasks. The resulting cultures were fed with the fresh media twice per week and almost all the neurons will die due to the high concentration of serum. Then the mixed glial cells were subjected to a series shake-off procedure to obtain pure OPCs two weeks later. Specifically, the mixed glial cells were shaken initially for 1 h at 100 rpm to remove microglia and shaken for 20-22 h at 37° C. at 200 rpm to enrich OPCs. OPCs were collected by centrifugation at 1200 rpm for 5 min, resuspended in basal chemically defined medium (BDM). BDM consisted of DMEM supplemented with N2 medium (Invitrogen), glutamine (4 mM), BSA (0.1 mg/ml), hydrocortisone (20 nM), selenium (30 nM) and biotin (10 nM). OPCs were plated on poly-ornithine (PO, 50 μg/ml for 1 hr)-coated culture dishes and maintained in BDM supplemented with bFGF (10 ng/ml) and PDGF (10 ng/ml) before experimental manipulation. OPCs were 95% pure judged by A2B5+ staining.

OPC Transfection

OPCs were transfected with siRNA with the protocol as follows: OPCs were digested with 0.05% trypsin-EDTA and pelleted at 100 g relative centrifugal force for 15 min. OPCs were resuspended in DMEM and pelleted again to rinse away all trypsin. A total of 2-3×10⁶ aliquot of OPCs was resuspended in 100 μl of Amaxa OPC nucleofection reagent (VPG-1009; Amaxa, Lonza) containing 100 pmol of SMARTpool rat Hrh3 (L-093904-01, Dharmacon) or 100 pmol of si-control nontargeting small interfering RNA (siRNA) pool (D-001810-10, Dharmacon) then were electroporated with the Amaxa nucleofection apparatus, using 0-17 program. Transfected OPCs were plated into PO-coated 6-well plate and cultured for 4 days in BDM supplemented with triiodothyronine (30 nM) and N-acetyl-L-cystenine (30 μM). The cells were harvested and protein samples were analyzed with western blot.

Reagents

The antibodies used for immunoblot experiments were rabbit anti-myelin basic protein (MBP, Millipore), mouse anti-myelin-associated glycoprotein (MAG, abcam) and rabbit anti-H3R (Millipore). All the siRNA smartpools are purchased from Dharmacon, Thermo.

Western Blot

After treatments, OPCs were washed twice with ice-cold PBS and total cell lysates were harvested in RIPA lysis buffer (50 mM Tris-HCl, pH 7.5, 1 mM EDTA, 1 mM EGTA, 1 mM sodium orthovanadate, 50 mM sodium fluoride, 0.1% 2-mercaptoethanol, 1% triton X-100 and proteases inhibitor cocktail). The lysates were briefly sonicated and stored at −80° C. before Western analysis. The BCA Protein Assay Reagent (PIERCE, Thermo) was performed to determine protein concentration. 15 g of total protein per sample was aliquoted, boiled for 5 min after mixed with 2×SDS buffer (125 mM Tris-HCl, 4% SDS, 20% glycerol, 100 mM DTT) and separated by SDS-PAGE on Bis-Tris mini-gels (Invitrogen). Separated proteins were then transferred to nitrocellulose membranes and blocked in 5% milk/TBS-0.1% Tween for 1 h at room temperature. Membranes were then incubated in the presence of primary antibodies diluted in 5% milk/TBS-0.1% Tween overnight at 4° C. The following day, membranes were washed 3 times for 5 min with TBS-0.1% Tween and incubated for 1 h at room temperature in 5% milk/TBS-0.1%

Tween containing goat anti-rabbit or goat anti-mouse secondary antibodies (Jackson ImmunoResearch Laboratories, West Grove, Pa.) at a dilution of 1:5000. The detection of HRP conjugated secondary antibodies was performed by enhanced chemiluminescence substrate mixture (PIERCE) using the FUJI imaging device (FUJI, Tokyo, Japan).

Quantitative Real-Time RT-PCR

Total RNA was isolated using an RNeasy Mini Kit (Qiagen) and first-strand cDNA was synthesized using a Sensiscript RT Kit (Qiagen) according to the manufacturer's instructions. mRNA expression was determined by real-time PCR using SYBR Green Master mix under standard thermocycler conditions (Applied Biosystems). Data were collected and quantitatively analyzed on an ABI Prism 7900 sequence detection system (Applied Biosystems). Sequences of PCR primer pairs were: Sequences of PCR primer pairs were: β-actin (internal control): forward 5′-GCGTCCACCCGCGAGTACAAC-3′, reverse 5′-CGACGACGAGCGCAGCGATA-3′; Hrh3: forward 5′-TACTGTGTGCCTCCTCGGTCTT-3′ and reverse 5′-AGCTCGAGTGACTGACAGGAATC-3′.

Statistical Analysis

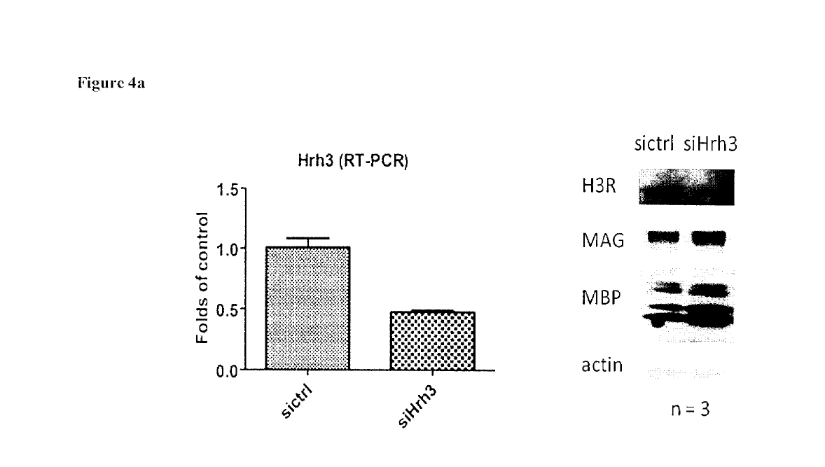

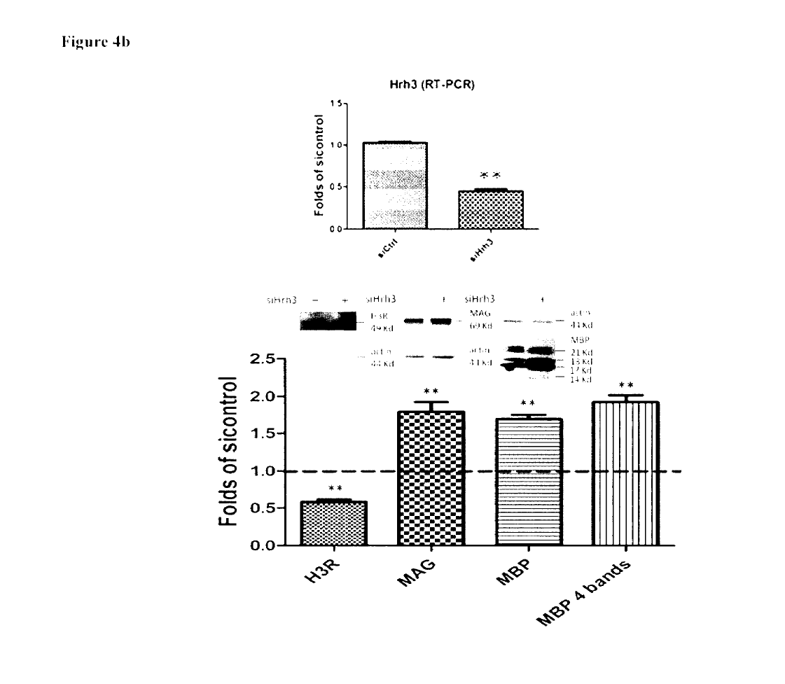

All data are given as mean±standard error of the mean (SEM). Statistical analysis in FIG. 4b was performed using one-way ANOVA or student's t test when appropriate. Statistical significance was inferred if p<0.05.

Results

To confirm the engagement of H3R in oligodendrocyte differentiation, specific siRNA was employed to knock down H3R expression. As shown in FIG. 4a, knockdown of H3R (down to ˜40% of normal, RT-PCR) led to increased expression levels of myelin basic protein (MBP) and myelin-associated glycoprotein (MAG), biomarkers of mature oligodendrocytes. Statistical analysis was performed and as shown in FIG. 4b, knockdown of H3R with siRNA significantly reduced expression of endogenous H3R, resulting in statistically significant increase in expression of two mature oligodendrocyte biomarkers: myelin basic protein MBP (the 18 kDa band), 1.7±0.1 fold of si-control, p<0.01. Additional bands of MBP (21, 17 and 14 kDa), which when measured together with the 18 kDa band demonstrated a 1.9±0.1 fold change compared to si-control p<0.01; and myelin-associated glycoprotein MAG, 1.8±0.2 fold of si-control, n=5), **, p<0.01, siHrh3 vs. si-control.

These findings suggest that H3R, as one of few GPCRs with constitutive activity, negatively regulates oligodendrocyte differentiation, which is consistent with the results shown in FIGS. 1 to 3.

### Conclusion

Reduction in H3R expression level by siRNA promoted OPC differentiation, as evident by increased expression of MBP and MAG. The result demonstrates a negative role of constitutive activity of H3R in oligodendrocyte differentiation.

Preclinical Experiments

All in vivo studies were conducted in compliance with Project Licences obtained, according to protocols approved by the Institutional Animal Care and Use Committee of GSK R&D China and in compliance with the GlaxoSmithKline company policy on the care and used of laboratory animals and related codes of practice.

### Example 11

The ability of the compound of Example 1 to enhance in vivo remyelination was determined with the mouse cuprizone-induced demyelination model.

Cuprizone Treatment

The cuprizone model was conducted with the protocol as follows: the C57BL/6 mice at age of 8 weeks were fed with powder mouse food mixed freshly with 0.2% cuprizone (w/w) for 5 weeks to induce demyelination, then animals were allowed to recover (removal of cuprizone from the diet) and administrated with Example compound 1, at 0.3 mg/kg, 1 mg/kg, 3 mg/kg and 10 mg/kg body weight orally, b.i.d. for an additional 9 days prior to sacrifice. The brain samples were collected for assessment of myelination. The compound of Example 1 was formulated as a suspension using 1% aqueous methylcellulose as the vehicle.

Myelin Staining and Quantification

Mice were deeply anesthetized and quickly perfused with 0.9% saline via the left cardiac ventricle to drain out blood. Whole brains were taken out and put into 15 ml tube with 4% paraformaldehyde (PFA) for post-fixation overnight, then soaked in 30% sucrose for 24-48 h to dehydrate. For frozen embedding, brains were immediately snap frozen in isopentane mixed with dry ice and then embedded in optimal cutting temperature solution (OCT). Whole brains were sectioned at coronal position from Bregma −2.46 mm to 1.18 mm according to mouse atlas by Paxinos and Franklin (The Mouse Brain in Stereotaxic Coordinates_{—}3rd Edition) with cryostat (MICROM HM525) at a thickness of 30 μm. All sections were put into 96-well plate filled with anti-freezing solution [300 g sucrose dissolved in 500 ml 0.1 M PB solution (combine 11.505 g Na₂HPO₄ and 2.275 g NaH₂PO₄, dilute to 1 L with distilled water, adjust pH to 7.4) plus 300 ml Ethylene glycol and dilute to 1 L with distilled water]. For Black-gold II staining, 2 sections from forebrain around the bregma 0.86 mm and 2 sections from hindbrain around the bregma −1.58 mm for each mouse respectively were chosen according to mouse atlas by Paxinos and Franklin. Black-gold II staining for detection of myelination was performed according to a protocol adapted from the manufacturer (AG105, Millipore). Briefly, 2 free-floating sections from forebrain and 2 sections from hindbrain were rehydrated 2× in MilliQ water for 2 minutes each time and then stained in a 24-well plate with 0.3% Black-gold II solution at 60° C. for 20 minutes. The sections were monitored to determine the extent of staining. When the finest myelinated fibers were stained dark red to black, the staining process was stopped. The sections were then rinsed 2× in MilliQ water for 2 minutes each time. Then the sections were treated with Sodium thiosulfate solution (1%) for 6 minutes at 60° C. After rinsing in PBST (0.05% Triton X100 in PBS), sections were mounted on slides (Leica Microsystems Plus Slides) and further air-dried on 37° C. heating platform for 2-4 hours. The dehydrated slides were coverslipped with robotic coverslipper (CV5030, Leica). Stained slides were scanned by Scanscope (Aperio Technologies Inc.). Digital images of central corpus callosum or cortex close to cingulum were captured using ImageScope (Aperio Technologies Inc.) at the magnification of 20×.

Image-Pro 6.3 software (Media Cybernetics, Bethesda, Md. 20814 USA) was used for subsequent quantitative evaluation. Threshold was set to Black-gold II staining in each gated corpus callosum and held constant for images obtained at equal objectives and light intensities on slides that were processed. Two parameters were used here: area and IOD (Integrated Optical Density). For measurement of demyelination area, all digital images were set under the same colour range after converted to a greyscale value of 8. Demyelination area indicated by void of Black-gold staining and total area of central corpus callosum at 20× magnification were measured separately on each image. Mean demyelination area was calculated as: (demyelination area/total area of central corpus callosum)×100. For measurement of mean density, areas and IOD in central corpus callosum or cortex were calculated after all digital images were set under the same colour range. Mean density was calculated as: IOD/total area of central corpus callosum or gated cortex. The result was expressed as percentage of mean density of naïve control. Data from two sections from forebrain, two sections from hindbrain, and four sections from both forebrain and hindbrain of each animal respectively were averaged and analyzed. Group data was expressed as mean±SEM. Graphs were generated by GraphPad5 PRISM software (GraphPad Software, Inco, San Diego, Calif., USA).

Statistical Analysis

Graphs were generated by GraphPad5 PRISM software (GraphPad Software, Inco, San Diego, Calif., USA). T-test by R software was used for analysis of the difference between groups. P value<0.05 was considered statistically significant. Significance is indicated in the figures by asterisks *p<0.05, **p<0.01.

Results

Effect of Example Compound 1, on Remyelination in the Cuprizone Model

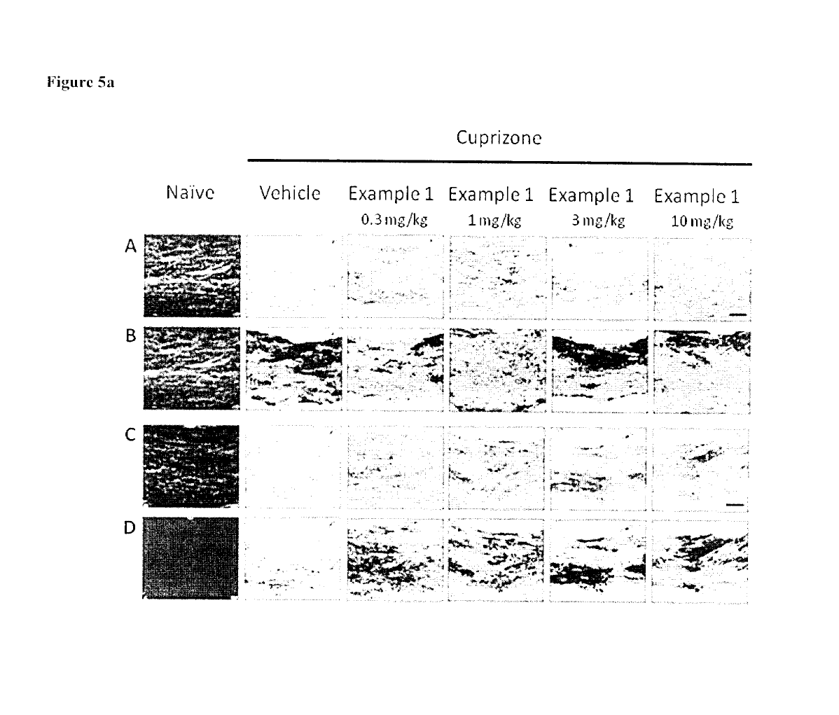

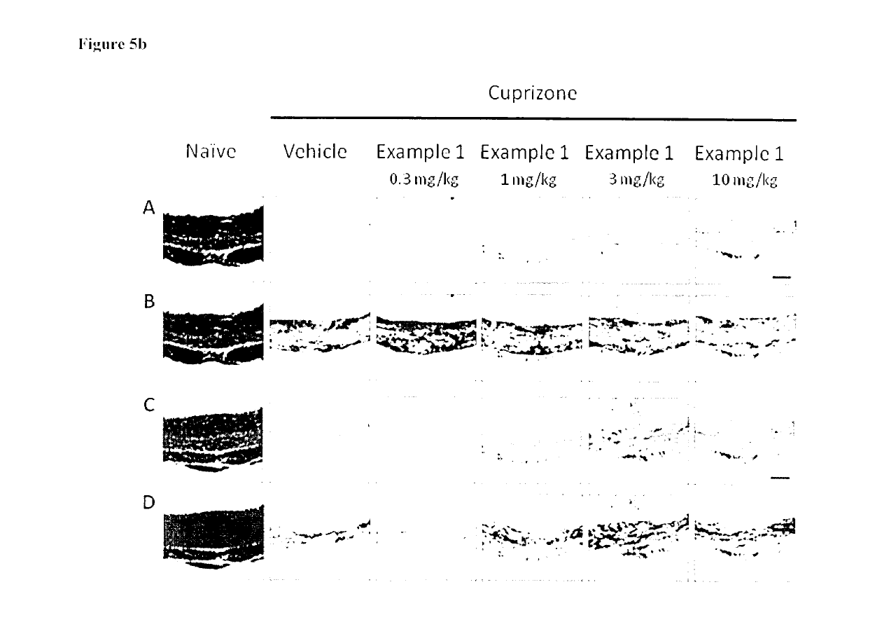

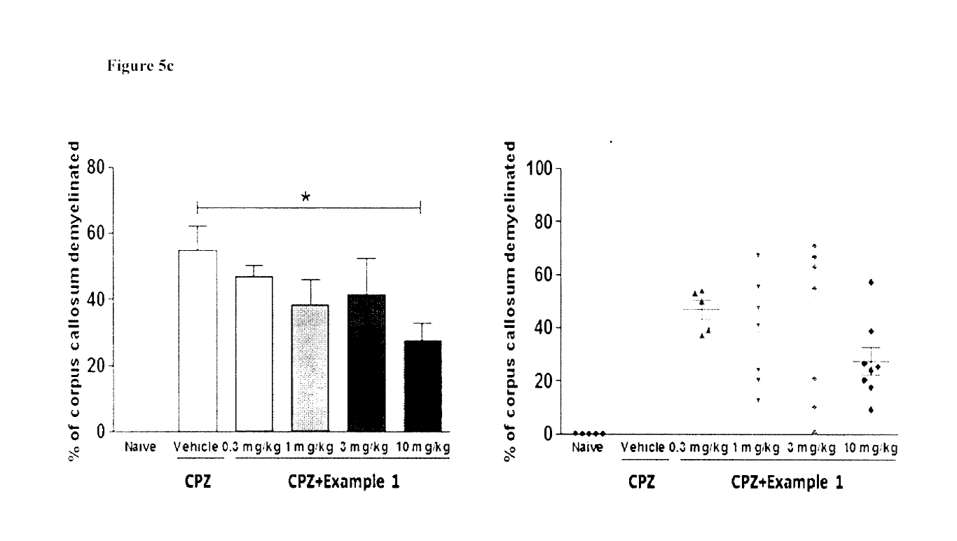

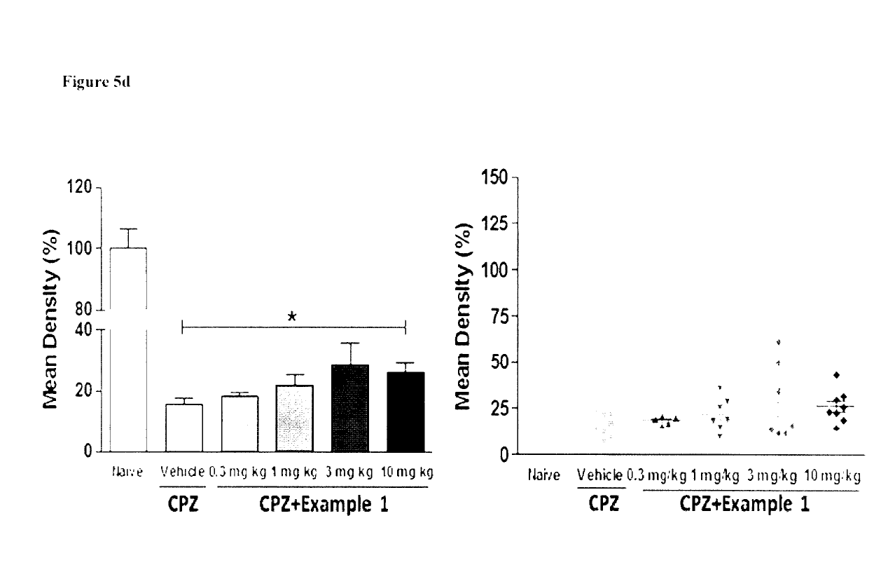

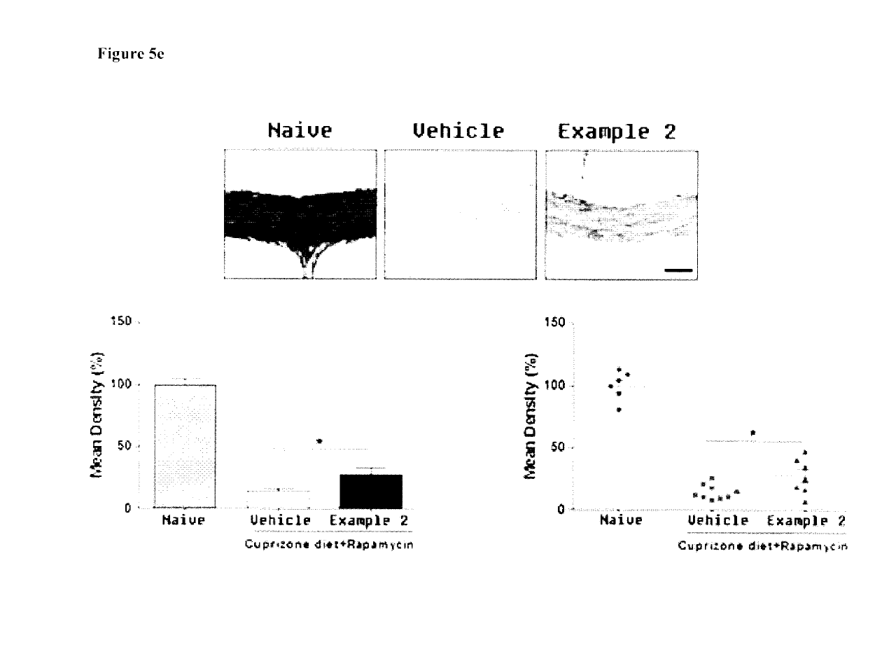

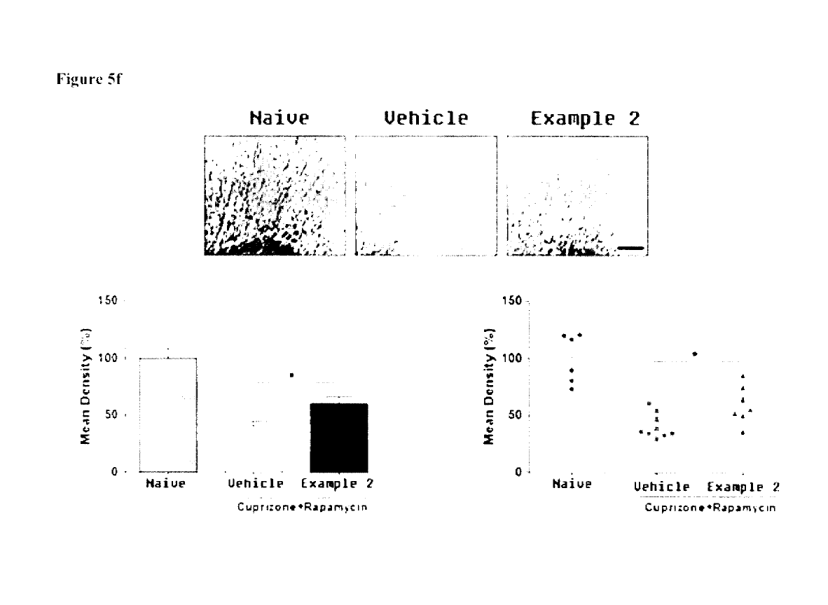

Treatment with 5 weeks of cuprizone diet followed by 9 days of normal diet led to a severe demyelination in corpus callosum, as visualized by the loss of Black-gold II staining in vehicle control group when compared with the naïve group (FIGS. 5a and 5b).

In vivo remyelination was determined by two different quantitative parameters, demyelination area and mean staining intensity indicated by Black-gold II staining. As shown in FIGS. 5a-5d, treatment with Example compound 1, (10 mg/kg, 9 days) significantly increased intensity of myelin-specific Black-gold II staining in the lesion and decreased the demyelination area of corpus callosum in both forebrain and hindbrain compared to vehicle control group; the effect of Example compound 1 increased with ascending doses (except 3 mg/kg).

### Example 12

The ability of Example compound 2 to enhance in vivo remyelination was determined with the mouse cuprizone/rapamycin-induced demyelination model.

Cuprizone Plus Rapamycin Treatment

The cuprizone plus rapamycin model was conducted with the protocol as follows: rapamycin was dissolved in 100% ethanol and stored at −20° C. until use. Immediately before injection, the rapamycin was diluted in vehicle solution to get a final concentration in 5% PEG-400, 5% Tween 80, and 4% ethanol. The C57BL/6 mice at age of 8 weeks were fed with powder mouse food mixed freshly with 0.2% cuprizone (w/w) and received daily intraperitoneal injection of rapamycin (10 mg/kg body weight) for 5 weeks to induce demyelination, then animals were allowed to recover (removal of cuprizone from the diet and rapamycin injection) and administrated with Example compound 2, at 30 mg/kg body weight orally, b.i.d. for an additional 9 days prior to sacrifice. The brain samples were collected for pathologic analysis. Example compound 2 was formulated as a suspension using 1% aqueous methylcellulose as the vehicle.

Myelin Staining and Quantification

Mice were deeply anesthetized and quickly perfused with 0.9% saline via the left cardiac ventricle to drain out blood. Whole brains were taken out and put into 15 ml tube with 4% paraformaldehyde (PFA) for post-fixation overnight, then soaked in 30% sucrose for 24-48 h to dehydrate. For frozen embedding, brains were immediately snap frozen in isopentane mixed with dry ice and then embedded in optimal cutting temperature solution (OCT). Whole brains were sectioned at coronal position from Bregma −2.46 mm to 1.18 mm according to mouse atlas by Paxinos and Franklin (The Mouse Brain in Stereotaxic Coordinates_{—}3rd Edition) with cryostat (MICROM HM525) at a thickness of 30 μm. All sections were put into 96-well plate filled with anti-freezing solution [300 g sucrose dissolved in 500 ml 0.1 M PB solution (combine 11.505 g Na₂HPO₄ and 2.275 g NaH₂PO₄, dilute to 1 L with distilled water, adjust pH to 7.4) plus 300 ml Ethylene glycol and dilute to 1 L with distilled water]. For Black-gold II staining, 4 sections from forebrain (2 from corpus callosum, 2 from cortex) around the bregma 0.86 mm for each mouse respectively were chosen according to mouse atlas by Paxinos and Franklin. Black-gold II staining for detection of myelination was performed according to a protocol adapted from the manufacturer (AG105, Millipore). Briefly, 4 free-floating sections from forebrain (2 for corpus callosum, 2 for cortex) were rehydrated 2× in MilliQ water for 2 minutes each time and then stained in a 24-well plate with 0.3% Black-gold II solution at 60° C. for 20 minutes. The sections were monitored to determine the extent of staining. When the finest myelinated fibers were stained dark red to black, the staining process was stopped. The sections were then rinsed 2× in MilliQ water for 2 minutes each time. Then the sections were treated with Sodium thiosulfate solution (1%) for 6 minutes at 60° C. After rinsing in PBST (0.05% Triton X100 in PBS), sections were mounted on slides (Leica Microsystems Plus Slides) and further air-dried on 37° C. heating platform for 2-4 hours. The dehydrated slides were coverslipped with robotic coverslipper (CV5030, Leica). Stained slides were scanned by Scanscope (Aperio Technologies Inc.). Digital images of central corpus callosum or cortex close to cingulum were captured using ImageScope (Aperio Technologies Inc.) at the magnification of 20×.

Image-Pro 6.3 software (Media Cybernetics, USA) was used for subsequent quantitative evaluation. Threshold was set to Black-gold II staining in each gated corpus callosum and held constant for images obtained at equal objectives and light intensities on slides that were processed. Two parameters were used here: area and IOD (Integrated Optical Density). For measurement of mean density, areas and IOD in central corpus callosum or cortex were calculated after all digital images were set under the same colour range. Mean density was calculated as: IOD/total area of central corpus callosum or gated cortex. The result was expressed as percentage of mean density of naïve control. Data from two sections from forebrain corpus callosum, two sections from forebrain cortex of each animal respectively were averaged and analyzed. Group data was expressed as mean±SEM. Graphs were generated by GraphPad5 PRISM software (GraphPad Software, Inco, USA).

Statistical Analysis

Graphs were generated by GraphPad5 PRISM software (GraphPad Software, Inc., USA). Unpaired t test by GraphPad was used for analysis of the difference between groups. P value<0.05 was considered statistically significant. Significance is indicated in the figures by asterisks *p<0.05, **p<0.01.

Effect of Example Compound 2 on Remyelination in the Cuprizone Plus Rapamycin Model

In another batch of cuprzione plus rapamycin model, mice were treated with cuprizone diet combined with intraperitoneal injections of rapamycin for 5 weeks followed by 9 days of compound administration. Cuprizone diet plus intraperitoneal injections of rapamycin induced severe demyelination in both corpus callosum and cortex and treatment with Example compound 2, (30 mg/kg, 9 days) significantly increased density of myelin specific Black-gold II staining in the lesion of corpus callosum and cortex in forebrain, compared to vehicle control group (FIGS. 5e and 5f).

Results (FIG. 5)

FIG. 5a Representative images show treatment with Example compound 1, decreased demyelinated areas and increased mean intensity of Black-gold II staining in forebrain corpus callosum in cuprizone model (5 weeks of cuprizone diet+9 days of compound treatment).

FIG. 5b Representative images show treatment with Example compound 1, decreased demyelinated areas and increased mean intensity of Black-gold II staining of in hindbrain corpus callosum in cuprizone model (5 weeks of cuprizone diet+9 days of compound treatment).

FIG. 5c shows statistical analysis of treatment effect of Example compound 1 on remyelination, as evidenced by reduction of demyelination area. Four sections from both forebrain and hindbrain for each animal were analyzed. Data for each group were expressed as Mean±SEM. **P<0.01 vs. vehicle control group.

FIG. 5d shows statistical analysis of treatment effect of Example compound 1, on remyelination, as indicated by increased mean density of Black-gold II staining in corpus callosum. Four sections from both forebrain and hindbrain for each animal were analyzed. Data for each group were expressed as Mean±SEM. *P<0.05 vs. vehicle control group.

FIG. 5e shows treatment with Example compound 2, promoted remyelination in forebrain corpus callosum in the cuprizone plus rapamycine model (5 weeks of cuprizone diet and rapamycin injection+9 days of compound treatment). Representative images (top panel) and quantification analysis (bottom panel) demonstrated that Example compound 2 treatment for 9 days in the recovery phase significantly increased mean density of Black-gold II staining in forebrain corpus callosum, compared to the vehicle control group. Two sections from forebrain corpus callosum for each animal were averaged and analyzed. Data for each group were expressed as Mean±SEM. *P<0.05 vs. vehicle group.

FIG. 5f shows treatment with Example compound 2, promoted remyelination in forebrain cortex in the cuprizone plus rapamycine model (5 weeks of cuprizone diet and rapamycin injection+9 days of compound treatment). Representative images (top panel) and quantification analysis (bottom panel) demonstrated that Example compound 2 treatment for 9 days in the recovery phase significantly increased mean density of Black-gold II staining in forebrain cortex, compared to the vehicle control group. Two sections from forebrain cortex for each animal were averaged and analyzed. Data for each group were expressed as Mean±SEM. *P<0.05 vs. vehicle group.

As shown in FIG. 5a, treatment with Example compound 1, decreased demyelinated areas (void of black-gold II staining) and increased mean intensity of Black-gold II staining (as indicated by representative images in A and C), compared to vehicle control, in forebrain corpus callosum in cuprizone model (5 weeks of cuprizone diet+9 days of compound treatment). B and D are examples to show how the analysis was conducted. B is a derived image from A transformed by Image-Pro 6.3 software (Media Cybernetics, USA), red area in B was measured as demyelination area. D is a derived image from A transformed by Image-Pro, intensity of red colour in D was measured as myelin intensity.

As shown in FIG. 5b, treatment with Example compound 1, decreased demyelinated areas (void of black-gold II staining) and increased mean intensity of Black-gold II staining (as indicated by representative images in A and C), compared to vehicle control, in hindbrain corpus callosum in cuprizone model (5 weeks of cuprizone diet+9 days of compound treatment). B and D are examples to show how the analysis was conducted. B is a derived image from A transformed by Image-Pro 6.3 software, red area in B was measured as demyelination area. D is a derived image from A transformed by Image-Pro, intensity of red colour in D was measured as myelin intensity.

As shown in FIG. 5c, quantitative analysis showed that treatment with Example compound 1, at 10 mg/kg significantly decreased demyelination areas (vehicle group: 54.81%±7.27%; 0.3 mg/kg group: 46.82%±3.54%; 1 mg/kg group: 38.45%±7.55%; 3 mg/kg group: 41.22%±11.14%; 10 mg/kg group: 27.62%±5.20%; data from 4 sections per animal were analyzed; 2 forebrain sections, 2 hindbrain sections).

As shown in FIG. 5d, quantitative analysis showed that treatment with Example compound 1, at 10 mg/kg increased myelin staining intensity of corpus callosum in both forebrain and hindbrain (vehicle group: 15.75%±2.08%; 0.3 mg/kg group: 18.61%±1.01%; 1 mg/kg group: 22.00%±3.41%; 3 mg/kg group: 28.32%±7.76%; 10 mg/kg group: 26.36%±3.10%; data from 4 sections per animal were analyzed; 2 forebrain sections, 2 hindbrain sections).

As shown in FIG. 5e, treatment with Example compound 2, at 30 mg/kg, promoted remyelination in forebrain corpus callosum in the cuprizone plus rapamycine model (5 weeks of cuprizone diet and rapamycin injection+9 days of compound treatment). Representative images (top panel) and quantitative analysis (bottom panel) demonstrated that Example compound 2 treatment for 9 days in the recovery phase significantly increased mean density of Black-gold II staining in forebrain corpus callosum, compared to the vehicle control group (Mean intensity: vehicle: 14.65±1.96%; cpd: 27.68±5.44%. vehicle, n=9, Example compound 2, n=7, analyzed data from 2 sections per animal).

As shown in FIG. 5f, treatment with Example compound 2, at 30 mg/kg, promoted remyelination in forebrain cortex in the cuprizone plus rapamycine model (5 weeks of cuprizone diet and rapamycin injection+9 days of compound treatment). Representative images (top panel) and quantitative analysis (bottom panel) demonstrated that Example compound 2 treatment for 9 days in the recovery phase significantly increased mean density of Black-gold II staining in forebrain cortex, compared to the vehicle control group (Mean intensity: vehicle: 40.79±3.60%; cpd: 60.05±6.28%. vehicle, n=9, Example compound 2, n=7, analyzed data from 2 sections per animal).

### Conclusion

These results indicated that treatment of compounds of the invention Example compound 1 and Example compound 2 can enhance endogenous remyelination, which aligned well with the in vitro findings. Thus, the in vivo and in vitro data presented here strongly supports inverse agonists of the H3R as a novel therapy for multiple sclerosis by promoting CNS myelin repair through increased OPC differentiation.

### Example 13

### Histamine H3R Functional Inverse Agonist Assay with OPCs (cAMP Assay)

Histamine receptor 3 (H3R), expressed on cell surface, is negatively coupled to adenylyl cyclase, which stimulates the formation of cyclic AMP (cAMP). In the absence of histamine, the constitutively active H3R would inhibit intracellular level of cAMP. Inverse agonists of H3R, which block constitutive activity of H3R, thus would raise the cAMP level. The ability of Example compound 2 to inhibit constitutive activity of H3R was determined in a cellular cAMP assay.

For each compound being assayed, OPCs were seeded in PO coated 96-well plate at a density of 20000 cells/well, and cultured in BDM with bFGF (10 ng/ml Petrotech) and PDGF (10 ng/ml Petrotech) for 24 hours. The cells were first treated with different concentration of example compound (30 nM to 3 M) for 30 minutes. Then the cells were stimulated with Forskolin (3 M, Sigma) in the presence of the example compound for 15 minutes. The cAMP concentrations were measured by the cAMP Chemiluminescent Immunoassay Kit (Invitrogen, Cat. No. C10557). The cells were lysed with lysis buffer (60 μL, Invitrogen Kit reagent) at 37° C. for 30 minutes. The lysate were transferred to pre-coated microplate (Invitrogen Kit reagent) and mixed with cAMP-AP (30 μL, Invitrogen Kit reagent) and cAMP antibody (60 μL, Invitrogen Kit reagent). After 1 hour incubation, the wells were washed with the washing buffer (Invitrogen Kit reagent) 5 times. CSPD® Substrate/Sapphire-II™ Enhancer solution (100 μL, Invitrogen Kit reagent) was added into each well and incubated for 30 minutes. Chemiluminescence signal was measured in a SpectraMax M5 Multi-Mode Microplate Readers (Molecular Devices) for 1 second per well.

Results

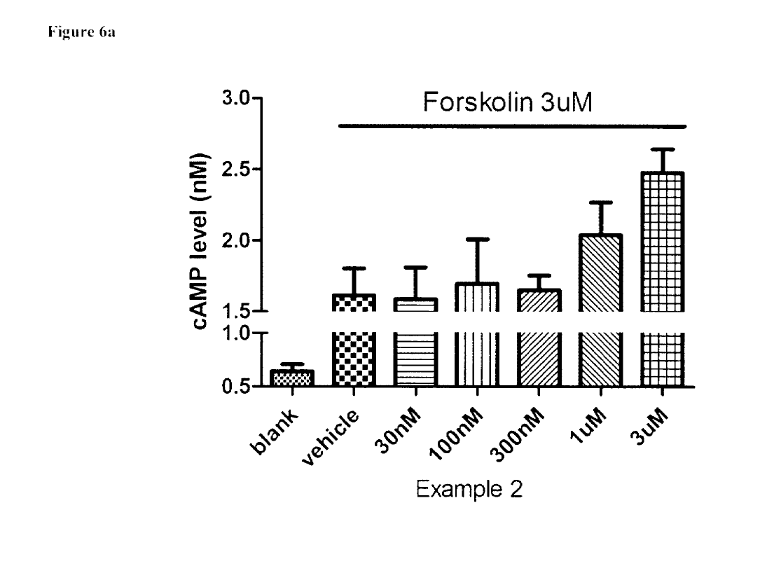

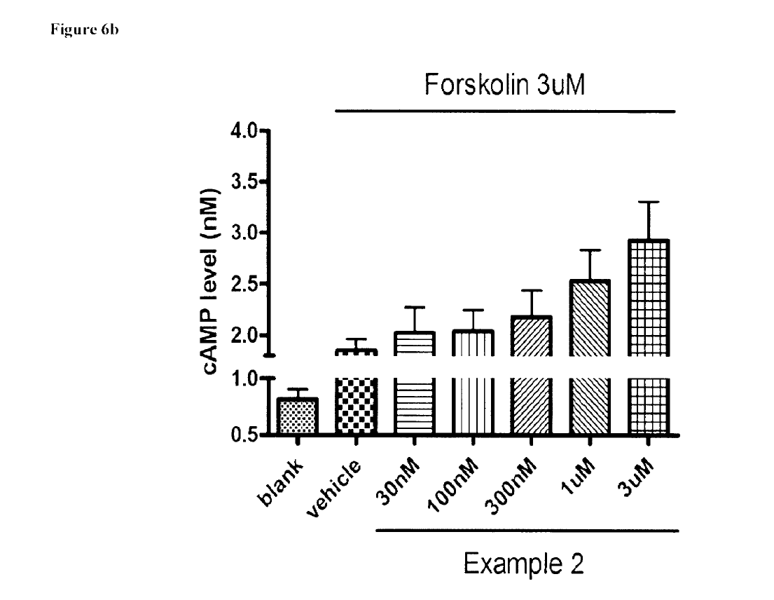

As shown in FIGS. 6a and 6b, Example compound 2 increased the Forskolin-stimulated cAMP level in the primary oligodendrocyte precursor cells in a dose-dependent manner.

### Conclusion

Example compound 2 increased intracellular forskolin-stimulated cAMP level in the oligodendrocyte precursor cells (in the absence of H3R agonist) in a dose-dependent manner. The results suggest that Example compound 2 is an inverse agonist of H3R.

### Example 14

The effect of Example compound 1 on basal GTPγS binding to the H3R receptor was determined in the GTPγS binding assay.

Cell Maintenance and Bacmam Virus Infection

Human embryonic kidney 293 cells stably expressing G protein GαO (HEK-293-GO) were maintained in minimum essential medium (MEM) supplemented with Earle's salts, 2 mM L-glutamine, 400 mg/ml geneticin and 10% foetal bovine serum at 37-8° C., 5% CO2 in a humidified environment. Exponentially growing cells were infected with BacMam virus encoding the human recombinant H3 receptor (Biocat: Virus 96801) as follows. Cells were detached from flasks in PBS and collected by centrifugation at 200×g for 5 min at room temperature. The cells were then resuspended in growth media containing virus at a multiplicity of infection (m.o.i.) of 100, re-plated and then incubated under normal growth conditions for 24 h.

GTPγS Binding Assay

GTPγS binding assays were performed using HEK-293-GαO cells transduced with human H3R-encoding BacMam virus as described above. Following overnight incubation, cells were collected into 10 ml PBS and spun at 200×g for 5 min. After removal of the supernatant, the pellet was resuspended and homogenised in 20 mM HEPES (pH 7.4) containing 3 mM MgCl₂, and 100 mM NaCl, centrifuged at 50,000×g for 20 min, then homogenised and centrifuged again. The membrane pellet was then resuspended and assayed for protein concentration.

Cell membranes were diluted to ˜1 mg/ml in assay buffer (20 mM HEPES, 100 mM NaCl, 10 mM MgCl2, pH 7.4) and incubated with wheat germ agglutinin scintillation proximity assay (SPA) beads (Amersham Biosciences) for 45 min, after which GDP (40 mM) was added. Various concentrations of example compound 1 (100 nM-0.001 nM in half log increments) were added to a 96-well plate along with 10 ml assay buffer. Non-specific binding was determined by the inclusion of 0.6 mM GTPγS. Sixty microliters (˜55 mg protein/well) of membranes/SPA beads/GDP mix was then added to each well and the plate incubated on an orbital shaker for 30 min at room temperature. [355]-GTPγS (0.3 nM) was then added to each well, the plate incubated on the shaker for a further 30 min, and incubations were stopped by rapid filtration under vacuum through Whatman GF/B filters. Filters were washed twice with 4 ml ice-cold water and bound [355]-GTPγS on the filters was measured by scintillation counting on a Wallac 1450 Microbeta Trilux counter.

Data Acquisition and Analysis

The data was analyzed and presented as fold change of the number of the vehicle control well (DMSO). Graphs were generated by softwares: Microsoft Excel and GraphPad Prism5.0. The curve fitting was performed with software GraphPad to derive the pEC₅₀ value of example compound 1. Curve fitting was performed by a Logistic Model embedded in the software (Sigmoidal dose-response: Y=Bottom+(Top−Bottom)/(1+10̂((Log EC50−X)))).

Statistical Analysis

All data are given as mean±standard error of the mean (SEM). Statistical analysis was performed using one-way ANOVA or student's t test when appropriate. Statistical significance was inferred if p<0.05.

Results

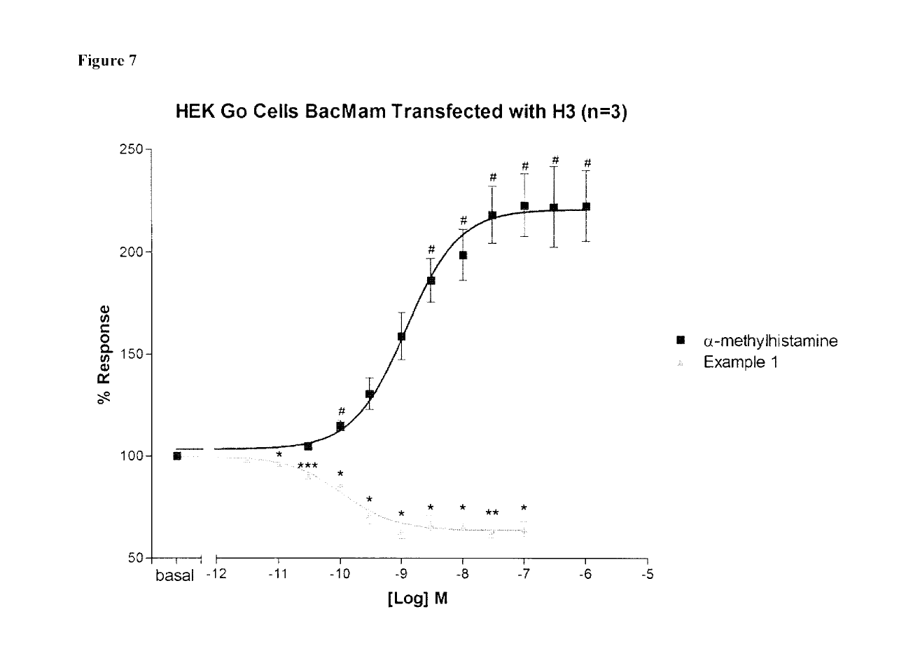

Example compound 1 exhibited inverse agonist properties at the human H3R expressed in HEK-293-GO cells. Basal GTPγS binding (in the absence of H3R agonist) was inhibited by Example compound 1 (100 nM-0.001 nM) in a dose-dependent manner with pEC50=9.95±0.07 from 3 independent experiments (FIG. 7). One-way analysis of variance (ANOVA) was used to compare the difference among the 11 dose groups (vehicle and 10 active doses). The p-value of ANOVA 9.735e-10<0.0001 indicated a statistically significant difference. α-methylhistamine, an H3R agonist, was used as a control to validate the assay. Data from 3 independent batches of GTPγS assay were averaged and expressed as fold change of the percentage in the vehicle control well. Example 1 vs vehicle, *, p<0.05, **, p<0.01, ***, p<0.001. α-methylhistamine vs vehicle, #, p<0.05.

### Conclusion

Example compound 1 inhibited basal GTPγS binding to H3R expressed on the membrane of HEK293 cells (in the absence of H3R agonist) in a dose-dependent manner. The results indicated that Example compound 1 is an inverse agonist of H3R.

1-5. (canceled)

6. A method of slowing, halting or reversing the progression of disability in MS, which comprises administering to the sufferer a therapeutically effective amount of a compound which is an inverse agonist of the H3 receptor.

7. A method according to claim 6, wherein the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

8. A method according to claim 6, wherein the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

9. A method according to claim 6, wherein the method comprises administering orally to a human the compound at a dosage of from 5 to 500 micrograms per day.

10. A method according to claim 6, wherein the method comprises administering orally to a human the compound at a dosage of from 10 to 150 micrograms per day.

11-13. (canceled)

14. (canceled)

15. A method of treating demyelinating diseases which comprises administering to the sufferer a therapeutically effective amount of a compound which is an inverse agonist of the H3 receptor, wherein the compound is 1-{6-[(3-cyclobutyl-2,3,4,5-tetrahydro-1H-3-benzazepin-7-yl)oxy]-3-pyridinyl}-2-pyrrolidinone, 3-(benzo[d][1,3]dioxol-5-yl)-5-((1-cyclobutylpiperidin-4-yl)methyl)-1,2,4-oxadiazole or 4-(4-((1-isopropylpiperidin-4-yl)oxy)piperidin-1-yl)benzonitrile; or pharmaceutically acceptable salts thereof.

16. A method according to claim 15, wherein the compound is 1-{6-[(3-cyclobutyl-2,3,4,5-tetrahydro-1H-3-benzazepin-7-yl)oxy]-3-pyridinyl}-2-pyrrolidinone, or a pharmaceutically acceptable salt thereof.

17-19. (canceled)

20. A pharmaceutical composition for oral administration to a human for use in the treatment of MS, comprising from 5 to 500 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof, and one or more pharmaceutically acceptable excipients.

21. A pharmaceutical composition according to claim 20, wherein the composition comprises from 10 to 150 micrograms of 1-{6-[(3-cyclobutyl-2,3,4,5-tetrahydro-1H-3-benzazepin-7-yl)oxy]-3-pyridinyl}-2-pyrrolidinone or a pharmaceutically acceptable salt thereof.

22. A compound which is 3-(benzo[d][1,3]dioxol-5-yl)-5-((1-cyclobutylpiperidin-4-yl)methyl)-1,2,4-oxadiazole, or a pharmaceutically acceptable salt thereof.

23-25. (canceled)

26. A method for treating MS which comprises administering orally to the sufferer a therapeutically effective amount of a compound which is an inverse agonist of the H3 receptor, wherein the compound is 1-(3-(3-(4-chlorophenyl)propoxy)-propyl)piperidine or (R)-6-(4-(3-(2-methylpyrrolidin-1-yl)propoxy)phenyl)pyridazin-3(2H)-one, or a pharmaceutically acceptable salt thereof.

27. A method according to claim 26, wherein the compound is 1434344-chlorophenyl)propoxy)-propyl)-piperidine or a pharmaceutically acceptable salt thereof.

28. A method according to claim 26, wherein the compound is (R)-6-(4-(3-(2-methylpyrrolidin-1-yl)propoxy)phenyl)pyridazin-3(2H)-one or a pharmaceutically acceptable salt thereof.

29-31. (canceled)

32. A method for treating MS which comprises administering orally to the sufferer a therapeutically effective amount of the compound according to claim 22.
